(19) United States
(12) Patent Application Publication
(10) Pub. No.: US 2015/0257739 A1
(43) Pub. Date: Sep. 17, 2015

(54) ULTRASOUND DIAGNOSTIC APPARATUS, IMAGE PROCESSING APPARATUS, AND IMAGE PROCESSING METHOD

(71) Applicants: Kabushiki Kaisha Toshiba, Minato-ku (JP); Toshiba Medical Systems Corporation, Otawara-shi (JP)

(72) Inventors: Cong YAO, Otawara (JP); Tetsuya Kawagishi, Nasushiobara (JP); Yoshitaka Mine, Nasushiobara (JP); Hiroki Yoshiara, Nasushiobara (JP); Shintaro Niwa, Nasushiobara (JP)

(73) Assignees: Kabushiki Kaisha Toshiba, Minato-ku (JP); Toshiba Medical Systems Corporation, Otawara-shi (JP)

(21) Appl. No.: 14/725,788

(22) Filed: May 29, 2015

Related U.S. Application Data
(63) Continuation-in-part of application No. PCT/JP2013/083776, filed on Dec. 17, 2013.

(30) Foreign Application Priority Data
Dec. 18, 2012 (JP) 2012-275981
Dec. 17, 2013 (JP) 2013-260340

(51) Int. Cl. A61B 8/08; A61B 8/00

(52) U.S. Cl. CPC A61B 8/481; A61B 8/5223; A61B 8/54; A61B 8/463; A61B 8/5207

(57) ABSTRACT

An ultrasound diagnostic apparatus according to an embodiment includes processing circuitry and controlling circuitry. The processing circuitry is configured to generate brightness transition information indicating a temporal transition of a brightness level in an analysis region that is set in an ultrasound scan region, from time-series data acquired by performing an ultrasound scan on a subject to whom a contrast agent has been administered and to obtain a parameter by normalizing reflux dynamics of the contrast agent in the analysis region with respect to time, based on the brightness transition information. The controlling circuitry is configured to cause a display to display the parameter in a format using one or both of an image and text.

### CROSS-REFERENCE TO RELATED APPLICATIONS

This application is a continuation-in-part of PCT international application Ser. No. PCT/JP2013/083776 filed on Dec. 17, 2013 which designates the United States, incorporated herein by reference, and which claims the benefit of priority from Japanese Patent Application No. 2012-275981, filed on Dec. 18, 2012 and Japanese Patent Application No. 2013-260340, filed on Dec. 17, 2013, the entire contents of which are incorporated herein by reference.

### FIELD

Embodiments described herein relate generally to an ultrasound diagnostic apparatus, an image processing apparatus, and an image processing method.

### BACKGROUND

In recent years, intravenously-administered ultrasound contrast agents have been available as products, so that “contrast echo methods” can be implemented. In the following sections, ultrasound contrast agents may simply be referred to as “contrast agents”. For example, one of the purposes of a contrast echo method is, when performing a medical examination on the heart or the liver, to inject a contrast agent through a vein so as to enhance bloodstream signals and to evaluate bloodstream dynamics. In many contrast agents, microbubbles function as reflection sources. For example, a second-generation ultrasound contrast agent called “Sonazoid (registered trademark)” that was recently launched in Japan includes microbubbles configured with phospholipid enclosing fluorocarbon (perfluorobutane) gas therein. When implementing the contrast echo method, it is possible to stably observe a reflux of the contrast agent, by using a transmission ultrasound wave having a medium-low sound pressure at such a level that does not destroy the microbubbles.

By performing an ultrasound scan on a diagnosed site (e.g., liver cancer) after administering the contrast agent thereto, an operator (e.g., a doctor) is able to observe an increase and a decrease of the signal strength, over a period of time from an inflow to an outflow of the contrast agent that refluxes due to the bloodstream. Further, studies have been made to perform a differential diagnosis process to determine benignancy/malignancy of a tumor region or to perform a diagnosis process on “diffuse” diseases, and the like, by observing differences in the temporal transition of the signal strength.

Unlike other simple morphological information, the temporal transition of the signal strength indicating reflux dynamics of a contrast agent usually requires that a moving image is interpreted in a real-time manner or after the moving image is recorded. Accordingly, it usually takes a long time to interpret the reflux dynamics of the contrast agent. For this reason, a method has been proposed by which information about the time at which a contrast agent flows in (inflow time), which is normally observed in a moving image, is mapped on a single still image. This method is realized by generating and displaying the still image in which the difference in the peak times of the signals of the contrast agent is expressed by using mutually-different hues. By referring to the still image, the interpreting doctor is able to easily understand the inflow time at each of the different locations on a tomographic plane of the diagnosed site. Further, another method has also been proposed by which a still image is generated and displayed so as to express, by using mutually-different hues, the difference in the times (the times from the start of an inflow to the end of an outflow) during which a contrast agent becomes stagnant in a specific region.

Incidentally, because tumor blood vessels run in a more complicated manner than normal blood vessels, phenomena may be observed in which microbubbles having no place to go become stagnant in a tumor or in which such stagnant microbubbles further flow in an opposite direction. Such behaviors of microbubbles inside tumor blood vessels were actually observed in tumor mice on which contrast enhanced ultrasound imaging processes were performed. In other words, if it is possible to evaluate behaviors of microbubbles by performing a contrast enhanced ultrasound imaging process which makes the imaging of a living body possible, there is a possibility that the contrast echo method may be applied to the evaluation of abnormalities of tumor blood vessels.

Further, in recent years, histopathological observations have confirmed that angiogenesis inhibitors, which are anticancer agents currently on a clinical trial, are able to destroy blood vessels that nourish a tumor so as to cause fragmentation and narrowing of the tumor blood vessels. If a contrast enhanced ultrasound imaging process is able to image or quantify the manner in which microbubbles become stagnant within blood vessels fragmented by an angiogenesis inhibitor, it is expected that the contrast echo method can be applied to judging effects of treatments.

However, the transition of the signal strength (i.e., the transition of brightness levels in an ultrasound image) varies depending on image taking conditions and measured regions. For example, the transition of the brightness levels varies depending on the type of the contrast agent, the characteristics of the blood vessels in the observed region, and the characteristics of the tissues in the surroundings of the blood vessels. In contrast, the above-mentioned still image is generated and displayed by determining a contrast agent inflow time on the basis of an absolute feature value (e.g., an absolute time or an absolute brightness level) that is observed regardless of the image taking conditions or the measured region and by analyzing the temporal transition of the signal strength on the basis of the determined contrast agent inflow time.

### BRIEF DESCRIPTION OF THE DRAWINGS

FIG. 1 is a block diagram of an exemplary configuration of an ultrasound diagnostic apparatus according to an embodiment;

FIG. 2, FIG. 3 and FIG. 4 are drawings of examples of an analysis region;

FIG. 5, FIG. 6, FIG. 7 and FIG. 8 are drawings for explaining an analyzing unit;

FIG. 9, FIG. 10 and FIG. 11 are drawings for explaining the transition image generating unit;

FIG. 12 is a flowchart of exemplary processes performed by the ultrasound diagnostic apparatus according to the present embodiment;

FIG. 13 and FIG. 14 are drawings for explaining modified examples of the present embodiment; and

FIG. 15 is a block diagram of an exemplary configuration of an ultrasound diagnostic apparatus according to a modified example.

### DETAILED DESCRIPTION

An ultrasound diagnostic apparatus according to an embodiment includes processing circuitry and controlling circuitry. The processing circuitry is configured to generate brightness transition information indicating a temporal transition of a brightness level in an analysis region that is set in an ultrasound scan region, from time-series data acquired by performing an ultrasound scan on a subject to whom a contrast agent has been administered and to obtain a parameter by normalizing reflux dynamics of the contrast agent in the analysis region with respect to time, based on the brightness transition information. The controlling circuitry is configured to cause a display to display the parameter in a format using one or both of an image and text.

An ultrasound diagnostic apparatus according to an embodiment includes a brightness transition information generating unit, an analyzing unit, and a controlling unit. The brightness transition information generating unit generates brightness transition information indicating a temporal transition of a brightness level in an analysis region that is set in an ultrasound scan region, from time-series data acquired by performing an ultrasound scan on a subject to whom a contrast agent has been administered. The analyzing unit obtains a parameter by normalizing reflux dynamics of the contrast agent in the analysis region with respect to time, based on the brightness transition information. The controlling unit causes a display unit to display the parameter in a format using one or both of an image and text.

Exemplary embodiments of an ultrasound diagnostic apparatus will be explained in detail below, with reference to the accompanying drawings.

### Exemplary Embodiments

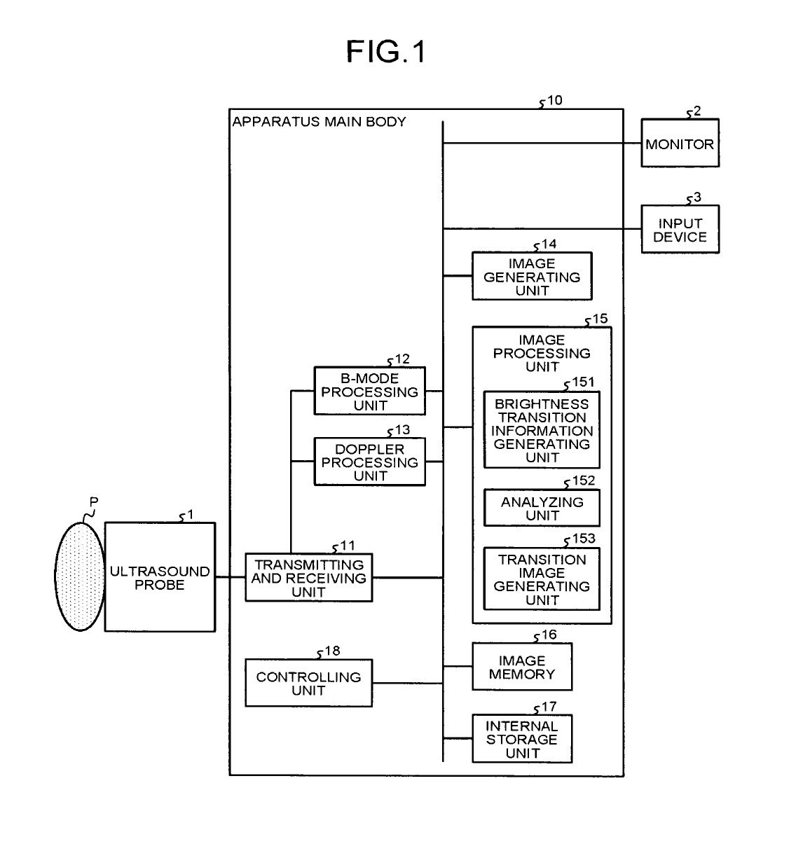

First, a configuration of an ultrasound diagnostic apparatus according to an exemplary embodiment will be explained. FIG. 1 is a block diagram of an exemplary configuration of the ultrasound diagnostic apparatus according to the present embodiment. As illustrated in FIG. 1, the ultrasound diagnostic apparatus according to the first embodiment includes an ultrasound probe 1, a monitor 2, an input device 3, and an apparatus main body 10.

The ultrasound probe 1 includes a plurality of piezoelectric transducer elements, which generate an ultrasound wave on the basis of a drive signal supplied from a transmitting and receiving unit 11 included in the apparatus main body 10 (explained later). Further, the ultrasound probe 1 receives a reflected wave from an examined subject (hereinafter, a “subject”) P and converts the received reflected wave into an electric signal. Further, the ultrasound probe 1 includes a matching layer that is abutted on the piezoelectric transducer elements, as well as a backing member that prevents backward propagation of ultrasound waves from the piezoelectric transducer elements. The ultrasound probe 1 is detachably connected to the apparatus main body 10.

When an ultrasound wave is transmitted from the ultrasound probe 1 to the subject P, the transmitted ultrasound wave is repeatedly reflected on discontinuous surfaces of acoustic impedances at a tissue in the body of the subject P and is received as a reflected-wave signal by the plurality of piezoelectric transducer elements included in the ultrasound probe 1. The amplitude of the received reflected-wave signal is dependent on the difference between the acoustic impedances on the discontinuous surfaces on which the ultrasound wave is reflected. When the transmitted ultrasound pulse is reflected on the surface of a flowing bloodstream, a cardiac wall, and the like, the reflected-wave signal is, due to the Doppler effect, subject to a frequency shift, depending on a velocity component of the moving members with respect to the ultrasound wave transmission direction.

For example, the apparatus main body 10 may be connected to a one-dimensional (1D) array probe which is served as the ultrasound probe 1 for a two-dimensional scan and in which the plurality of piezoelectric transducer elements are arranged in a row. Alternatively, for example, the apparatus main body 10 may be connected to a mechanical four-dimensional (4D) probe or a two-dimensional (2D) array probe which is served as the ultrasound probe 1 for a three-dimensional scan. The mechanical 4D probe is able to perform a two-dimensional scan by employing a plurality of piezoelectric transducer elements arranged in a row like in the 1D array probe and is also able to perform the three-dimensional scan by causing the plurality of piezoelectric transducer elements to swing at a predetermined angle (a swinging angle). The 2D array probe is able to perform the three-dimensional scan by employing a plurality of piezoelectric transducer elements arranged in a matrix formation and is also able to perform a two-dimensional scan by transmitting ultrasound waves in a focused manner.

The present embodiment is applicable to a situation where the ultrasound probe 1 performs a two-dimensional scan on the subject P and to a situation where the ultrasound probe 1 performs a three-dimensional scan on the subject P.

The input device 3 includes a mouse, a keyboard, a button, a panel switch, a touch command screen, a foot switch, a trackball, a joystick, and the like. The input device 3 receives various types of setting requests from an operator of the ultrasound diagnostic apparatus and transfers the received various types of setting requests to the apparatus main body 10. For example, from the operator, the input device 3 receives a setting of an analysis region used for analyzing the reflux dynamics of an ultrasound contrast agent. The analysis region set in the present embodiment will be explained in detail later.

The monitor 2 displays a Graphical User Interface (GUI) used by the operator of the ultrasound diagnostic apparatus to input the various types of setting requests through the input device 3, an ultrasound image, and the like generated by the apparatus main body 10.

The apparatus main body 10 is an apparatus that generates ultrasound image data on the basis of the reflected-wave signal received by the ultrasound probe 1. The apparatus main body 10 illustrated in FIG. 1 is an apparatus that is able to generate two-dimensional ultrasound image data on the basis of two-dimensional reflected-wave data received by the ultrasound probe 1. Further, the apparatus main body 10 illustrated in FIG. 1 is an apparatus that is able to generate three-dimensional ultrasound image data on the basis of three-dimensional reflected-wave data received by the ultrasound probe 1. In the following sections, three-dimensional ultrasound image data may be referred to as “volume data”.

As illustrated in FIG. 1, the apparatus main body 10 includes the transmitting and receiving unit 11, a B-mode processing unit 12, a Doppler processing unit 13, an image generating unit 14, an image processing unit 15, an image memory 16, an internal storage unit 17, and a controlling unit 18.

The transmitting and receiving unit 11 includes a pulse generator, a transmission delaying unit, a pulser, and the like and supplies the drive signal to the ultrasound probe 1. The pulse generator repeatedly generates a rate pulse for forming a transmission ultrasound wave at a predetermined rate frequency. Further, the transmission delaying unit applies a delay period that is required to focus the ultrasound wave generated by the ultrasound probe 1 into the form of a beam and to determine transmission directionality and that corresponds to each of the piezoelectric transducer elements, to each of the rate pulses generated by the pulse generator. Further, the pulser applies a drive signal (a drive pulse) to the ultrasound probe 1 with timing based on the rate pulses. In other words, the transmission delaying unit arbitrarily adjusts the transmission directions of the ultrasound waves transmitted from the piezoelectric transducer elements surface, by varying the delay periods applied to the rate pulses.

The transmitting and receiving unit 11 has a function to be able to instantly change the transmission frequency, the transmission drive voltage, and the like, for the purpose of executing a predetermined scanning sequence on the basis of an instruction from the controlling unit 18 (explained later). In particular, the configuration to change the transmission drive voltage is realized by using a linear-amplifier-type transmitting circuit of which the value can be instantly switched or by using a mechanism electrically switching among a plurality of power source units.

The transmitting and receiving unit 11 includes a pre-amplifier, an Analog/Digital (A/D) converter, a reception delaying unit, an adder, and the like and generates reflected-wave data by performing various types of processes on the reflected-wave signal received by the ultrasound probe 1. The pre-amplifier amplifies the reflected-wave signal for each of channels. The A/D converter applies an A/D conversion to the amplified reflected-wave signal. The reception delaying unit applies a delay period required to determine reception directionality to the result of the A/D conversion. The adder performs an adding process on the reflected-wave signals processed by the reception delaying unit so as to generate the reflected-wave data. As a result of the adding process performed by the adder, reflected components from the direction corresponding to the reception directionality of the reflected-wave signals are emphasized. A comprehensive beam used in an ultrasound transmission/reception is thus formed according to the reception directionality and the transmission directionality.

When a two-dimensional scan is performed on the subject P, the transmitting and receiving unit 11 causes the ultrasound probe 1 to transmit two-dimensional ultrasound beams. The transmitting and receiving unit 11 then generates two-dimensional reflected-wave data from the two-dimensional reflected-wave signals received by the ultrasound probe 1. When a three-dimensional scan is performed on the subject P, the transmitting and receiving unit 11 causes the ultrasound probe 1 to transmit three-dimensional ultrasound beams. The transmitting and receiving unit 11 then generates three-dimensional reflected-wave data from the three-dimensional reflected-wave signals received by the ultrasound probe 1.

Output signals from the transmitting and receiving unit 11 can be in a form selected from various forms. For example, the output signals may be in the form of signals called Radio Frequency (RF) signals that contain phase information or may be in the form of amplitude information obtained after an envelope detection process.

The B-mode processing unit 12 receives the reflected-wave data from the transmitting and receiving unit 11 and generates data (B-mode data) in which the strength of each signal is expressed by a degree of brightness, by performing a logarithmic amplification, an envelope detection process, and the like on the received reflected-wave data.

The B-mode processing unit 12 is capable of changing the frequency band to be imaged by changing a detection frequency by a filtering process. By using this function of the B-mode processing unit 12, it is possible to realize a contrast echo method, e.g., a Contrast Harmonic Imaging (CHI) process. In other words, from the reflected-wave data of the subject P into whom an ultrasound contrast agent has been injected, the B-mode processing unit 12 is able to separate reflected wave data (harmonic data or subharmonic data) of which the reflection source is microbubbles and reflected-wave data (fundamental harmonic data) of which the reflection source is tissues inside the subject P. Accordingly, by extracting the harmonic data or the subharmonic data from the reflected-wave data of the subject P, the B-mode processing unit 12 is able to generate B-mode data used for generating contrast enhanced image data. The B-mode data used for generating the contrast enhanced image data is such data in which the strength of each reflected-wave signal of which the reflection source is the contrast agent is expressed by a degree of brightness. Further, by extracting the fundamental harmonic data from the reflected-wave data of the subject P, the B-mode processing unit 12 is able to generate B-mode data used for generating tissue image data.

When performing a CHI process, the B-mode processing unit 12 is able to extract harmonic components by using a method different from the method described above that uses the filtering process. During the harmonic imaging process, it is possible to implement any of the imaging methods including an Amplitude Modulation (AM) method, a Phase Modulation (PM) method, and an AMPM method combining the AM method with the PM method. According to the AM method, the PM method, or the AMPM method, a plurality of ultrasound transmission is performed with respect to the same scanning line (multiple rates), while varying the amplitude and/or the phase. As a result, the transmitting and receiving unit 11 generates and outputs a plurality of pieces of reflected-wave data for each of the scanning lines. After that, the B-mode processing unit 12 extracts the harmonic components by performing an addition/subtraction process depending on the modulation method on the plurality of pieces of reflected-wave data for each of the scanning lines. After that, the B-mode processing unit 12 generates B-mode data by performing an envelope detection process or the like on the reflected-wave data of the harmonic components.

For example, when implementing the PM method, the transmitting and receiving unit 11 causes ultrasound waves having mutually-the-same amplitude and inverted phase polarities (e.g., (−1, 1)) to be transmitted twice for each of the scanning lines, according to a scan sequence set by the controlling unit 18. After that, the transmitting and receiving unit 11 generates reflected-wave data resulting from the “−1” transmission and reflected-wave data resulting from the “1” transmission. The B-mode processing unit 12 adds these two pieces of reflected-wave data. As a result, a signal from which fundamental harmonic components are eliminated and in which second harmonic components primarily remain is generated. After that, the B-mode processing unit 12 generates CHI B-mode data (the B-mode data used for generating contrast enhanced image data), by performing an envelope detection process or the like on the generated signal. The CHI B-mode data is such data in which the strength of each reflected-wave signal of which the reflection source is the contrast agent is expressed by a degree of brightness. When implementing the PM method with a CHI process, for example, the B-mode processing unit 12 is able to generate the B-mode data used for generating tissue image data, by performing a filtering process on the reflected-wave data resulting from the “1” transmission.

The Doppler processing unit 13 obtains velocity information from the reflected-wave data received from the transmitting and receiving unit 11 by performing a frequency analysis, extracts bloodstream, tissues, and contrast-agent echo components under the influence of the Doppler effect, and generates data (Doppler data) obtained by extracting moving member information such as a velocity, a dispersion, a power, and the like, for a plurality of points.

The B-mode processing unit 12 and the Doppler processing unit 13 according to the present embodiment are able to process both two-dimensional reflected-wave data and three-dimensional reflected-wave data. In other words, the B-mode processing unit 12 is able to generate two-dimensional B-mode data from two-dimensional reflected-wave data and to generate three-dimensional B-mode data from three-dimensional reflected-wave data. The Doppler processing unit 13 is able to generate two-dimensional Doppler data from two-dimensional reflected-wave data and to generate three-dimensional Doppler data from three-dimensional reflected-wave data.

The image generating unit 14 generates ultrasound image data from the data generated by the B-mode processing unit 12 and the Doppler processing unit 13. In other words, from the two-dimensional B-mode data generated by the B-mode processing unit 12, the image generating unit 14 generates two-dimensional B-mode image data in which the strength of the reflected wave is expressed by a degree of brightness. Further, from the two-dimensional Doppler data generated by the Doppler processing unit 13, the image generating unit 14 generates two-dimensional Doppler image data expressing the moving member information. The two-dimensional Doppler image data is a velocity image, a dispersion image, a power image, or an image combining these images.

In this situation, generally speaking, the image generating unit 14 converts (by performing a scan convert process) a scanning line signal sequence from an ultrasound scan into a scanning line signal sequence in a video format used by, for example, television and generates display-purpose ultrasound image data. Specifically, the image generating unit 14 generates the display-purpose ultrasound image data by performing a coordinate transformation process compliant with the ultrasound scanning used by the ultrasound probe 1. Further, as various types of image processes other than the scan convert process, the image generating unit 14 performs, for example, an image process (a smoothing process) to re-generate a brightness-average image or an image process (an edge enhancement process) using a differential filter within images, while using a plurality of image frames obtained after the scan convert process is performed. Further, the image generating unit 14 superimposes text information of various parameters, scale marks, body marks, and the like on the ultrasound image data.

In other words, the B-mode data and the Doppler data are the ultrasound image data before the scan convert process is performed. The data generated by the image generating unit 14 is the display-purpose ultrasound image data obtained after the scan convert process is performed. The B-mode data and the Doppler data may also be referred to as raw data.

Further, the image generating unit 14 generates three-dimensional B-mode image data by performing a coordinate transformation process on the three-dimensional B-mode data generated by the B-mode processing unit 12. Further, the image generating unit 14 generates three-dimensional Doppler image data by performing a coordinate transformation process on the three-dimensional Doppler data generated by the Doppler processing unit 13. In other words, the image generating unit 14 generates “the three-dimensional B-mode image data or the three-dimensional Doppler image data” as “three-dimensional ultrasound image data (volume data)”.

Further, the image generating unit 14 performs a rendering process on the volume data, to generate various types of two-dimensional image data used for displaying the volume data on the monitor 2. Examples of the rendering process performed by the image generating unit 14 include a process to generate Multi Planar Reconstruction (MPR) image data from the volume data by implementing an MPR method. Other examples of the rendering process performed by the image generating unit 14 include a process to apply a “curved MPR” to the volume data and a process to apply a “maximum intensity projection” to the volume data. Another example of the rendering process performed by the image generating unit 14 is a Volume Rendering (VR) process to generate two-dimensional image data reflecting three-dimensional information.

The image memory 16 is a memory that stores therein the display-purpose image data generated by the image generating unit 14. Further, the image memory 16 is also able to store therein the data generated by the B-mode processing unit 12 or the Doppler processing unit 13. After a diagnosis process, for example, the operator is able to invoke the display-purpose image data stored in the image memory 16. Further, after a diagnosis process, for example, the operator is also able to invoke the B-mode data or the Doppler data stored in the image memory 16, and the invoked data is served as the display-purpose ultrasound image data by the image generating unit 14. Further, the image memory 16 is also able to store data output from the transmitting and receiving unit 11.

The image processing unit 15 is installed in the apparatus main body 10 for performing a Computer-Aided Diagnosis (CAD) process. The image processing unit 15 obtains data stored in the image memory 16 and performs image processes thereon to support diagnosis processes. Further, the image processing unit 15 stores results of the image processes into the image memory 16 or the internal storage unit 17 (explained later). Processes performed by the image processing unit 15 will be described in detail later.

The internal storage unit 17 stores therein various types of data such as a control computer program (hereinafter, “control program”) to execute ultrasound transmissions and receptions, image process, and display process, as well as diagnosis information (e.g., patients' IDs, doctors' observations), diagnosis protocols, and various types of body marks. Further, the internal storage unit 17 may be used, as necessary, for storing therein any of the image data stored in the image memory 16. Further, it is possible to transfer the data stored in the internal storage unit 17 to an external apparatus by using an interface (not shown). Examples of the external apparatus include various types of medical image diagnostic apparatuses, a personal computer (PC) used by a doctor who performs an image diagnosis process, a storage medium such as a compact disk (CD) or a digital versatile disk (DVD), and a printer.

The controlling unit 18 controls the entire processes performed by the ultrasound diagnostic apparatus. Specifically, on the basis of the various types of setting requests input by the operator by the input device 3 and various types of control programs and various types of data invoked from the internal storage unit 17, the controlling unit 18 controls processes performed by the transmitting and receiving unit 11, the B-mode processing unit 12, the Doppler processing unit 13, the image generating unit 14, and the image processing unit 15. Further, the controlling unit 18 exercises control so that the monitor 2 displays the image data stored in the image memory 16 and the internal storage unit 17.

An overall configuration of the ultrasound diagnostic apparatus according to the present embodiment has thus been explained. The ultrasound diagnostic apparatus according to the present embodiment configured as described above implements the contrast echo method for the purpose of analyzing the reflux dynamics of the contrast agent. Further, from time-series data acquired by performing an ultrasound scan on the subject P into whom an ultrasound contrast agent has been administered, the ultrasound diagnostic apparatus according to the present embodiment generates and displays image data with which it is possible to analyze, by using objective criteria, the reflux dynamics of the contrast agent in an analysis region that is set in the ultrasound scan region.

To generate the image data, the image processing unit 15 according to the present embodiment includes, as illustrated in FIG. 1, a brightness transition information generating unit 151, an analyzing unit 152, and a transition image generating unit 153.

The brightness transition information generating unit 151 illustrated in FIG. 1 generates brightness transition information indicating a temporal transition of brightness levels in an analysis region that is set in an ultrasound scan region, from time-series data acquired by performing an ultrasound scan on the subject P into whom a contrast agent has been administered. Specifically, as the brightness transition information, the brightness transition information generating unit 151 generates a brightness transition curve that is a curve indicating the temporal transition of the brightness levels in the analysis region. As long as the information is able to reproduce a brightness transition curve, the brightness transition information generating unit 151 may generate the brightness transition information in any arbitrary form. The time-series data described above may be represented by a plurality of pieces of two- or three-dimensional contrast enhanced image data generated in time series by the image generating unit 14 during a contrast enhanced time. Alternatively, the time-series data described above may be represented by a plurality of pieces of two- or three-dimensional harmonic data (harmonic components) extracted in time series by the B-mode processing unit 12 during a contrast enhanced time. Alternatively, the time-series data described above may be represented by a plurality of pieces of two- or three-dimensional B-mode data generated in time series by the B-mode processing unit 12 during a contrast enhanced time for the purpose of generating contrast enhanced image data.

In other words, when a contrast enhanced imaging process is performed in a two-dimensional ultrasound scan region, the brightness transition information generating unit 151 generates a brightness transition curve for a two-dimensional analysis region that is set in a two-dimensional scan region, from time-series data acquired by performing a two-dimensional scan on the subject P. In contrast, when a contrast enhanced imaging process is performed in a three-dimensional ultrasound scan region, the brightness transition information generating unit 151 generates a brightness transition curve for a three- or two-dimensional analysis region that is set in a three-dimensional scan region, from time-series data acquired by performing a three-dimensional scan on the subject P.

In the following sections, an example will be explained in which the brightness transition information generating unit 151 generates a brightness transition curve for a two-dimensional analysis region that is set in a two-dimensional scan region, from a plurality of pieces of contrast enhanced image data acquired in time series by performing a two-dimensional scan on the subject P.

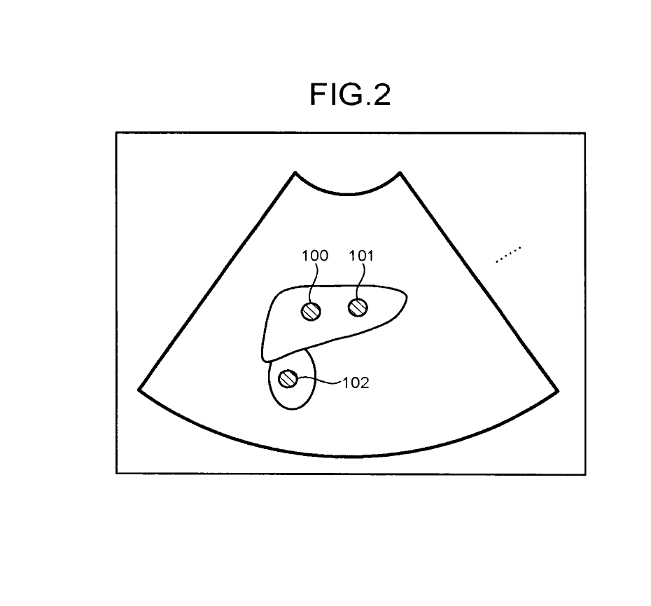

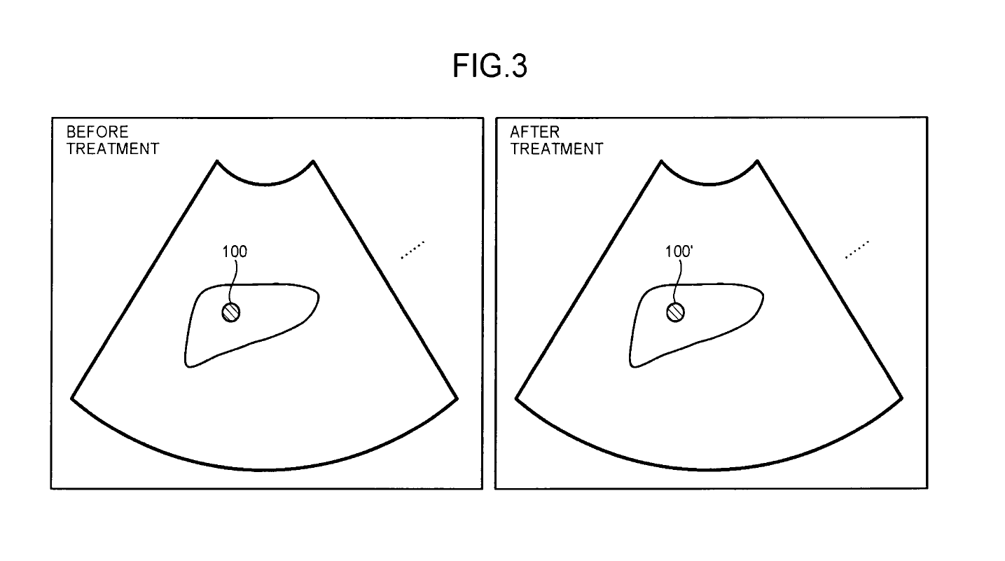

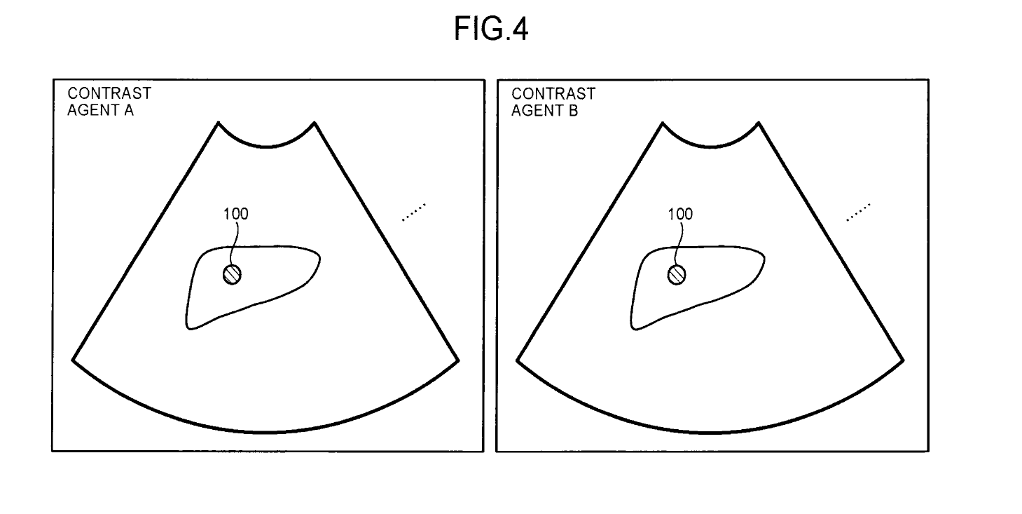

In this situation, the brightness transition information generating unit 151 according to the present embodiment generates a plurality of brightness transition curves. For example, the brightness transition information generating unit 151 may generate the plurality of brightness transition curves respectively for a plurality of analysis regions that are set in an ultrasound scan region. Alternatively, the brightness transition information generating unit 151 may generate the plurality of brightness transition curves for at least one mutually-the-same analysis region set in mutually-the-same ultrasound scan region, respectively from a plurality of pieces of time-series data acquired by performing an ultrasound scan in mutually-the-same ultrasound scan region during a plurality of mutually-different times. FIGS. 2, 3, and 4 are drawings of examples of the analysis region. In the following explanation, the position of the ultrasound probe 1 is fixed in the same location before and after the analysis region is set.

For example, as illustrated in FIG. 2, the operator sets an analysis region 100 in a tumor site in the liver, sets an analysis region 101 at the portal vein of the liver, and sets an analysis region 102 in a kidney, the liver and the kidney being rendered in B-mode image data (tissue image data) before a contrast enhancement. The analysis region 101 is set for the purpose of comparing dynamics of the bloodstream that refluxes in the tumor site with dynamics of the bloodstream that refluxes in the entire liver. Further, normally, the liver is dyed by the contrast agent, after the kidney is dyed. For this reason, the analysis region 102 is set for the purpose of comparing dynamics of the bloodstream that refluxes in the entire liver with dynamics of the bloodstream that refluxes in the entire kidney.

After the analysis regions 100 to 102 are set, the brightness transition information generating unit 151 calculates an average brightness level in the analysis region 100, an average brightness level in the analysis region 101, and an average brightness level in the analysis region 102, from each of a plurality of pieces of contrast enhanced image data acquired in time series. From the calculation results, the brightness transition information generating unit 151 generates three brightness transition curves.

Alternatively, as illustrated in the left section of FIG. 3, for example, the operator sets an analysis region 100 in B-mode image data before a contrast enhancement, before performing a treatment using an angiogenesis inhibitor. The brightness transition information generating unit 151 generates a brightness transition curve of the analysis region 100 by calculating an average brightness level in the analysis region 100 from each of a plurality of pieces of contrast enhanced image data acquired in time series after the analysis region 100 is set.

Further, as illustrated in the right section of FIG. 3, for example, the operator sets an analysis region 100′ in the B-mode image data before the contrast enhancement so as to be in the same position as the analysis region 100, after performing the treatment using the angiogenesis inhibitor. The brightness transition information generating unit 151 generates a brightness transition curve of the analysis region 100′ by calculating an average brightness level in the analysis region 100′ from each of a plurality of pieces of contrast enhanced image data acquired in time series after the analysis region 100′ is set. The brightness transition curve of the analysis region 100 is served as a brightness transition curve before the treatment, whereas the brightness transition curve of the analysis region 100′ is served as a brightness transition curve after the treatment. The brightness transition information generating unit 151 has thus generated the two brightness transition curves.

Alternatively, as illustrated in the left section of FIG. 4, for example, at first, the operator sets an analysis region 100 in B-mode image data (tissue image data) before a contrast enhancement and performs a contrast enhanced imaging process using a contrast agent A. The brightness transition information generating unit 151 generates a brightness transition curve of the analysis region 100 with the contrast agent A by calculating an average brightness level in the analysis region 100 from each of a plurality of pieces of contrast enhanced image data acquired in time series after the analysis region 100 is set.

Further, for example, after a predetermined period (e.g., 10 minutes) has elapsed, the operator performs a contrast enhanced imaging process using a contrast agent B that is of a different type from the contrast agent A, as illustrated in the right section of FIG. 4. The brightness transition information generating unit 151 generates a brightness transition curve of the analysis region 100 with the contrast agent B by calculating an average brightness level in the analysis region 100 from each of a plurality of pieces of contrast enhanced image data acquired in time series after the contrast agent B is administered. The brightness transition information generating unit 151 has thus generated the two brightness transition curves.

With reference to FIGS. 3 and 4, the examples are explained in which the brightness transition curve of mutually-the-same single analysis region is generated from each of the two pieces of time-series data acquired during the mutually-different times. The present embodiment, however, is also applicable to a situation where brightness transition curves of mutually-the-same multiple analysis regions are generated from each of the two pieces of time-series data acquired during the mutually-different times. Further, the present embodiment is also applicable to a situation where there are three or more pieces of time-series data acquired during mutually-different times.

The analyzing unit 152 illustrated in FIG. 1 obtains a parameter by normalizing reflux dynamics of the contrast agent in the analysis region with respect to time, based on the brightness transition information. In this situation, the analyzing unit 152 is able to obtain a parameter by normalizing the reflux dynamics of the contrast agent in the analysis region with respect to either the brightness levels or the brightness levels and time. In the present embodiment, an example will be explained in which the analyzing unit 152 obtains the parameter by normalizing the reflux dynamics of the contrast agent in the analysis region with respect to the brightness levels and time, based on the brightness transition information. In other words, the analyzing unit 152 obtains the parameter in which the reflux dynamics of the contrast agent in the analysis region are normalized, by analyzing the shape of each of the brightness transition curves. Specifically, the analyzing unit 152 generates a normalized curve from each of the brightness transition curves by normalizing either a time axis or a brightness axis and the time axis. In the present embodiment, the analyzing unit 152 generates the normalized curves from the brightness transition curves by normalizing the brightness axis and the time axis. For example, to generate the normalized curves, the analyzing unit 152 obtains, in each of the brightness transition curves, a maximum point at which the brightness level exhibits a maximum value, a first point at which the brightness level reaches, before the maximum point, a first multiplied value obtained by multiplying the maximum value by a first ratio, and a second point at which the brightness level reaches, after the maximum point, a second multiplied value obtained by multiplying the maximum value by a second ratio. The first ratio and the second ratio may be initially set or may be set in advance by the operator. The first ratio and the second ratio may arbitrarily be changed by the operator.

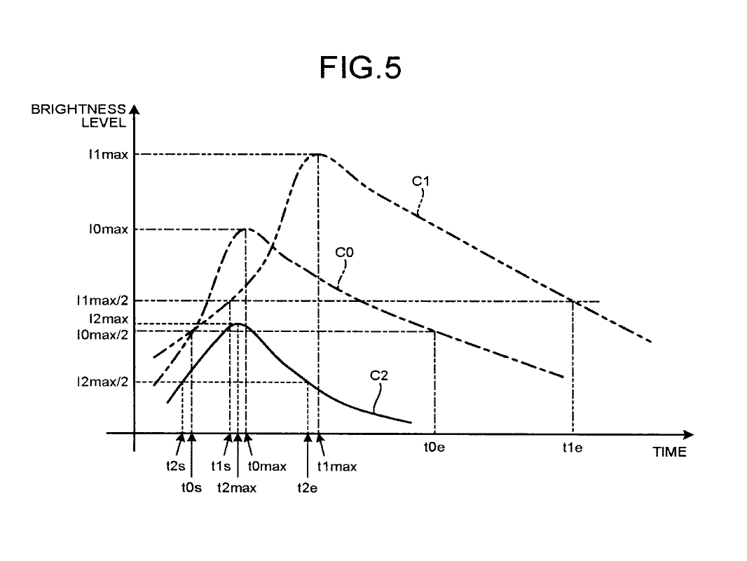

Next, processes performed by the analyzing unit 152 while using the brightness transition curves of the analysis regions 100 to 102 illustrated in FIG. 2 will be explained with reference to FIGS. 5 to 8. FIGS. 5 to 8 are drawings for explaining the analyzing unit.

In FIG. 5, the brightness transition curve of the analysis region 100 is shown as a curve C0 (the one-dot dashed line), while the brightness transition curve of the analysis region 101 is shown as a curve C1 (the two-dot dashed line), and the brightness transition curve of the analysis region 102 is shown as a curve C2 (the solid line). The brightness transition curves illustrated in FIG. 5 are approximate curves generated by the brightness transition information generating unit 151 from the time-series data of the average brightness levels in the analysis regions, while using a mathematical model. In the following sections, an example in which the first ratio and the second ratio are both set to “50%” will be explained. The present embodiment is also applicable to a situation where the first ratio and the second ratio are set to different values from each other (e.g., 20% and 30%).

As illustrated in FIG. 5, the analyzing unit 152 analyzes the curve C0 and obtains the maximum point “time: t0max; brightness level: I0max”. Further, the analyzing unit 152 calculates a value “I0max/2” that is equal to half of the maximum brightness level. After that, as illustrated in FIG. 5, the analyzing unit 152 obtains, in the curve C0, the first point “time: t0s; brightness level: I0max/2” at which the brightness level reaches “I0max/2” before the maximum time. In addition, as illustrated in FIG. 5, the analyzing unit 152 obtains, in the curve C0, the second point “time: toe; brightness level: I0max/2” at which the brightness level reaches “I0max/2” after the maximum time.

By performing a similar process, as illustrated in FIG. 5, the analyzing unit 152 analyzes the curve C1 and obtains the maximum point “time: t1max; brightness level: I1max”, the first point “time: t1s; brightness level: I1max/2”, and the second point “time: t1e; brightness level: I1max/2”. Further, by performing a similar process, as illustrated in FIG. 5, the analyzing unit 152 analyzes the curve C2 and obtains the maximum point “time: t2max; brightness level: I2max”, the first point “time: t2s; brightness level: I2max/2”, and the second point “time: t2e; brightness level: I2max/2”.

In this situation, the analyzing unit 152 determines “the time at the maximum point” to be a “maximum time” at which the contrast agent flowed into the analysis region at the maximum. Further, the analyzing unit 152 assumes “the time at the first point” to be the time at which the contrast agent started flowing into the analysis region and determines the time to be a “start time” at which the analysis of the dynamics of the bloodstream is started. In other words, the analyzing unit 152 sets the start time on the basis of the time it takes for the brightness level to decrease from the maximum value to the predetermined ratio (the first ratio), in the backward direction of the time axis of the brightness transition curve. In other words, the analyzing unit 152 sets the start time by calculating a threshold value (the first multiplied value) corresponding to the shape of the brightness transition curve served as an analysis target, by using mutually-the-same objective criterion (the first ratio). The start time is a time that is set by going back into the past after the maximum time is determined, i.e., a time that is set in a “retrospective” manner.

Further, the analyzing unit 152 assumes “the time at the second point” to be the time at which the contrast agent finished flowing out of the analysis region and determines the time to be an “end time” at which the analysis of the dynamics of the bloodstream is ended. In other words, the analyzing unit 152 sets the end time on the basis of the time it takes for the brightness level to decrease from the maximum value to the predetermined ratio (the second ratio), in the forward direction of the time axis of the brightness transition curve. In other words, the analyzing unit 152 sets the end time by calculating a threshold value (the second multiplied value) corresponding to the shape of the brightness transition curve served as an analysis target, by using mutually-the-same objective criterion (the second ratio). The end time is a time that is forecasted at the point in time when the maximum time is determined, i.e., a time that is set in a “prospective” manner.

Further, the analyzing unit 152 generates the normalized curves by normalizing the brightness transition curves, by using at least two points selected from these three points. After that, in the present embodiment, the analyzing unit 152 obtains a normalized parameter from the generated normalized curves. In this situation, to obtain a parameter related to the contrast agent inflow, the analyzing unit 152 generates a normalized curve by using the first point and the maximum point. As another example, to obtain a parameter related to the contrast agent outflow, the analyzing unit 152 generates a normalized curve by using the maximum point and the second point. As yet another example, to obtain a parameter related to the contrast agent inflow and the contrast agent outflow, the analyzing unit 152 generates a normalized curve by using the first point, the maximum point, and the second point.

In the present embodiment, because the plurality of brightness transition curves are generated, the analyzing unit 152 generates a normalized curve from each of the plurality of brightness transition curves. After that, in the present embodiment, the analyzing unit 152 obtains a parameter from each of the plurality of generated normalized curves. In the following sections, an example of a method for generating a normalized curve from each of the plurality of brightness transition curves by normalizing the brightness axis and the time axis will be explained.

First, a situation in which the parameter related to the contrast agent inflow is obtained will be explained. In that situation, the analyzing unit 152 generates a plurality of normalized curves respectively from the plurality of brightness transition curves, by setting a normalized time axis and a normalized brightness axis, on which the first points are plotted at a normalized first point that is mutually the same among the brightness transition curves and on which the maximum points are plotted at a normalized maximum point that is mutually the same among the brightness transition curves.

Specifically, the analyzing unit 152 obtains a brightness width and a time width between the first point and the maximum point from each of the brightness transition curves. After that, the analyzing unit 152 changes the scale of the brightness axis of each of the brightness transition curves in such a manner that the obtained brightness widths become equal to a constant value. Further, the analyzing unit 152 changes the scale of the time axis of each of the brightness transition curves in such a manner that the obtained time widths become equal to a constant value. After that, on the scale-changed brightness axis and the scale-changed time axis, the analyzing unit 152 sets the first points of the brightness transition curves at the normalized first point at the same coordinates and sets the maximum points of the brightness transition curves at the normalized maximum point at the same coordinates. Thus, the analyzing unit 152 has set the normalized time axis and the normalized brightness axis. After that, the analyzing unit 152 generates the plurality of normalized curves respectively from the plurality of brightness transition curves, by re-plotting the points structuring the curve from the first point to the maximum point in each of the brightness transition curves, on the normalized time axis and the normalized brightness axis.

For example, the analyzing unit 152 obtains “I0max/2”, “I1max/2”, and “I2max/2” from the curves C0, C1, and C2 illustrated in FIG. 5, respectively. Further, for example, the analyzing unit 152 obtains “t0max−t0s=t0r”, “t1max−t1s=t1r”, and “t2max−t2s=t2r”, from the curves C0, C1, and C2 illustrated in FIG. 5, respectively. After that, for example, as illustrated in FIG. 6, the analyzing unit 152 arranges “I0max/2, I1max/2, and I2max/2” each to be “50”. Further, for example, as illustrated in FIG. 6, the analyzing unit 152 arranges “t0max−t0s=t0r, t1max−t1s=t1r, and t2max−t2s=t2r” each to be “100”. Thus, the analyzing unit 152 has determined the scales of the normalized time axis and the normalized brightness axis.

After that, the analyzing unit 152 determines the coordinate system of the normalized time axis and the normalized brightness axis in such a manner that, for example, the first point on each of the curves C0 to C2 is at the normalized first point “normalized time: −100; normalized brightness level: 50” and that the maximum point on each of the curves C0 to C2 is at the normalized maximum point “normalized time: 0; normalized brightness level: 100”. Thus, the analyzing unit 152 has completed the process of setting the normalized time axis and the normalized brightness axis. After that, the analyzing unit 152 generates a normalized curve NC0(in) illustrated in FIG. 6, by re-plotting the points structuring the curve from the first point to the maximum point in the curve C0, on the normalized time axis and the normalized brightness axis. Similarly, the analyzing unit 152 generates a normalized curve NC1(in) illustrated in FIG. 6, from the curve C1. Similarly, the analyzing unit 152 generates a normalized curve NC2(in) illustrated in FIG. 6, from the curve C2.

Secondly, a situation in which the parameter related to the contrast agent outflow is obtained will be explained. In that situation, the analyzing unit 152 generates a plurality of normalized curves respectively from the plurality of brightness transition curves, by setting a normalized time axis and a normalized brightness axis, by which the maximum points are plotted at a normalized maximum point that is mutually the same among the brightness transition curves and by which the second points are plotted at a normalized second point that is mutually the same among the brightness transition curves.

Specifically, the analyzing unit 152 obtains a brightness width and a time width between the maximum point and the second point from each of the brightness transition curves. After that, the analyzing unit 152 changes the scale of the brightness axis of each of the brightness transition curves in such a manner that the obtained brightness widths become equal to a constant value. Further, the analyzing unit 152 changes the scale of the time axis of each of the brightness transition curves in such a manner that the obtained time widths become equal to a constant value. After that, by using the scale-changed brightness axis and the scale-changed time axis, the analyzing unit 152 sets the maximum points of the brightness transition curves at the normalized maximum point at the same coordinates and sets the second points of the brightness transition curves at the normalized second point at the same coordinates. Thus, the analyzing unit 152 has set the normalized time axis and the normalized brightness axis. After that, the analyzing unit 152 generates the plurality of normalized curves respectively from the plurality of brightness transition curves, by re-plotting the points structuring the curve from the maximum point to the second point in each of the brightness transition curves, on the normalized time axis and the normalized brightness axis.

For example, the analyzing unit 152 obtains “I0max/2”, “I1max/2”, and “I2max/2” from the curves C0, C1, and C2 illustrated in FIG. 5, respectively. In the present embodiment, because the first ratio and the second ratio are the same ratio, the brightness width between the maximum point and the second point is the same value as the brightness width between the maximum point and the first point, for each of the brightness transition curves. Further, for example, the analyzing unit 152 obtains “t0e−t0max=t0p”, “t1e−t1max=t1p”, and “t2e−t2max=t2p”, from the curves C0, C1, and C2 illustrated in FIG. 5, respectively. After that, for example, as illustrated in FIG. 7, the analyzing unit 152 arranges “I0max/2, I1max/2, and I2max/2” each to be “50”. Further, for example, as illustrated in FIG. 7, the analyzing unit 152 arranges “t0e-t0max=t0p, t1e−t1max=t1p, and t2e-t2max=t2p”, each to be “100”. Thus, the analyzing unit 152 has determined the scales of the normalized time axis and the normalized brightness axis.

After that, the analyzing unit 152 determines the coordinate system of the normalized time axis and the normalized brightness axis in such a manner that, for example, the maximum point in each of the curves C0 to C2 is at the normalized maximum point “normalized time: 0; normalized brightness level: 100” and that the second point in each of the curves C0 to C2 is at the normalized second point “normalized time: 100; normalized brightness level: 50”. Thus, the analyzing unit 152 has completed the process of setting the normalized time axis and the normalized brightness axis. After that, the analyzing unit 152 generates a normalized curve NC0(out) illustrated in FIG. 7, by re-plotting the points structuring the curve from the maximum point to the second point in the curve C0, on the normalized time axis and the normalized brightness axis. Similarly, the analyzing unit 152 generates a normalized curve NC1(out) illustrated in FIG. 7, from the curve C1. Similarly, the analyzing unit 152 generates a normalized curve NC2(out) illustrated in FIG. 7, from the curve C7.

Thirdly, a situation in which the parameter related to the contrast agent inflow and the contrast agent outflow is obtained will be explained. In that situation, the analyzing unit 152 generates a plurality of normalized curves respectively from the plurality of brightness transition curves, by setting a normalized time axis and a normalized brightness axis, by which the first points, the maximum points, and the second points are plotted at the normalized first point, the normalized maximum point, and the normalized second point, respectively, on the brightness transition curves.

Specifically, the analyzing unit 152 obtains a brightness width (a first brightness width) and a time width (a first time width) between the first point and the maximum point from each of the brightness transition curves. Further, the analyzing unit 152 obtains a brightness width (a second brightness width) and a time width (a second time axis) between the maximum point and the second point from each of the brightness transition curves. After that, the analyzing unit 152 changes the scale of the brightness axis of each of the brightness transition curves in such a manner that the first brightness widths of the brightness transition curves become equal to a constant value (dI1) and that the second brightness widths of the brightness transition curves become equal to another constant value (dI2). In this situation, the analyzing unit 152 ensures that “dI1:dI2=the first ratio:the second ratio” is satisfied. Further, the analyzing unit 152 changes the scale of the time axis of each of the brightness transition curves in such a manner that the first time widths of the brightness transition curves become equal to a constant value (dT1) and that the second time widths of the brightness transition curves become equal to another constant value (dT2). In this situation, the analyzing unit 152 ensures that “dT1:dT2=the first ratio:the second ratio” is satisfied.

After that, by using the scale-changed brightness axis and the scale-changed time axis, the analyzing unit 152 sets the first points of the brightness transition curves at the normalized first point at the same coordinates, sets the maximum points of the brightness transition curves at the normalized maximum point at the same coordinates, and sets the second points of the brightness transition curves at the normalized second point at the same coordinates. For example, if the first ratio is “20%”, and the second ratio is “30%”, the coordinates of the normalized first point is set at “normalized time: −100; normalized brightness level: 20”, while the coordinates of the normalized maximum point is set at “normalized time: 0; normalized brightness level: 100”, and the coordinates of the normalized second point is set at “normalized time: 150; normalized brightness level: 30”.

Thus, the analyzing unit 152 has set the normalized time axis and the normalized brightness axis. After that, the analyzing unit 152 generates the plurality of normalized curves respectively from the plurality of brightness transition curves, by re-plotting the points structuring the curve from the first point to the second point via the maximum point in each of the brightness transition curves, on the normalized time axis and the normalized brightness axis.

In the present embodiment, because the first ratio and the second ratio are both “50%”, the analyzing unit 152 generates a normalized curve NC0 illustrated in FIG. 8, by combining the normalized curve NC0(in) and the normalized curve NC0(out) that are generated from the curve C0. Similarly, the analyzing unit 152 generates a normalized curve NC1 illustrated in FIG. 8, by combining the normalized curve NC1(in) and the normalized curve NC1(out) that are generated from the curve C1. Similarly, the analyzing unit 152 generates a normalized curve NC2 illustrated in FIG. 8, by combining the normalized curve NC2(in) and the normalized curve NC2(out) that are generated from the curve C2.

From the normalized curves described above, the analyzing unit 152 obtains normalized parameters. For example, the analyzing unit 152 obtains, from the normalized curves, a normalized time at which the normalized brightness level is “80” and a normalized brightness level at which the normalized time is “50”, as the normalized parameters.

After that, the controlling unit 18 causes the monitor 2 to display the parameters (the normalized parameters) in a format using either an image or text. The display mode of the parameters may be selected from various modes; however, in the present embodiment, an example in which the parameters are displayed in a format using an image will be explained. Specifically, in the following sections, an example will be explained in which a parametric imaging is performed by using the parameters obtained from the normalized curves, as one of the display modes using an image (an image format). A display mode of the parameters in a format using text and a display mode of the parameters in a format using an image other than the parametric imaging will be explained in detail later.

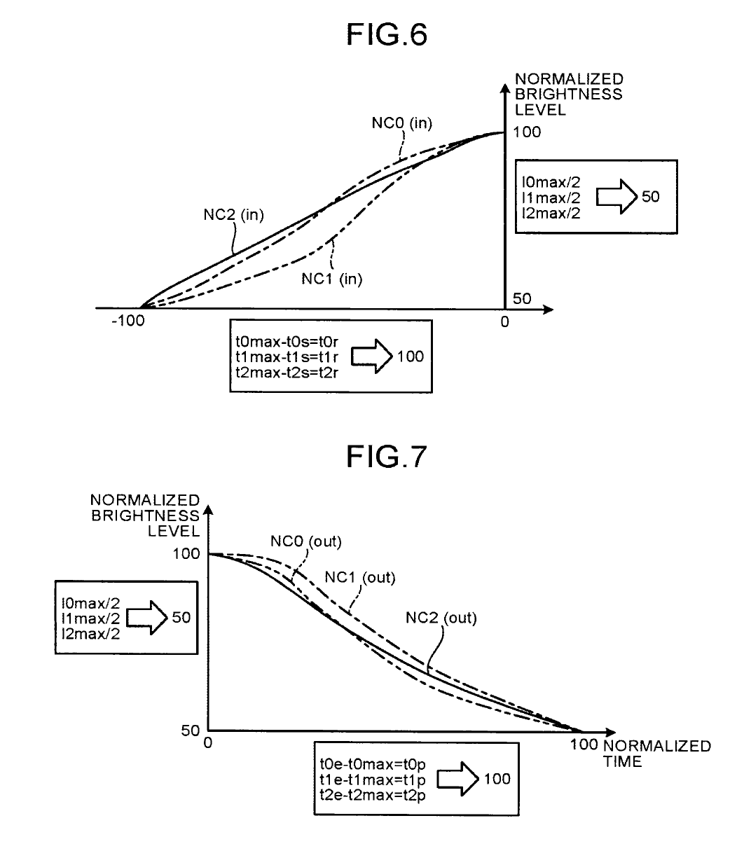

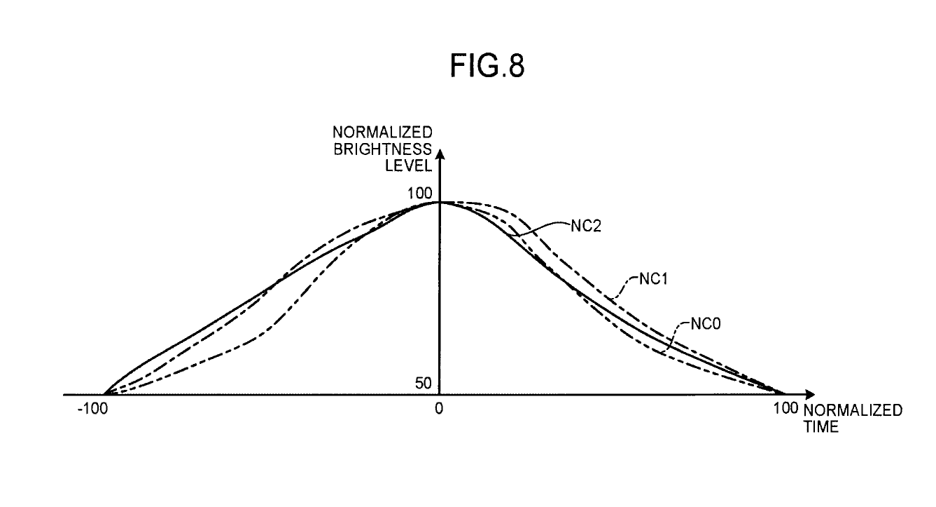

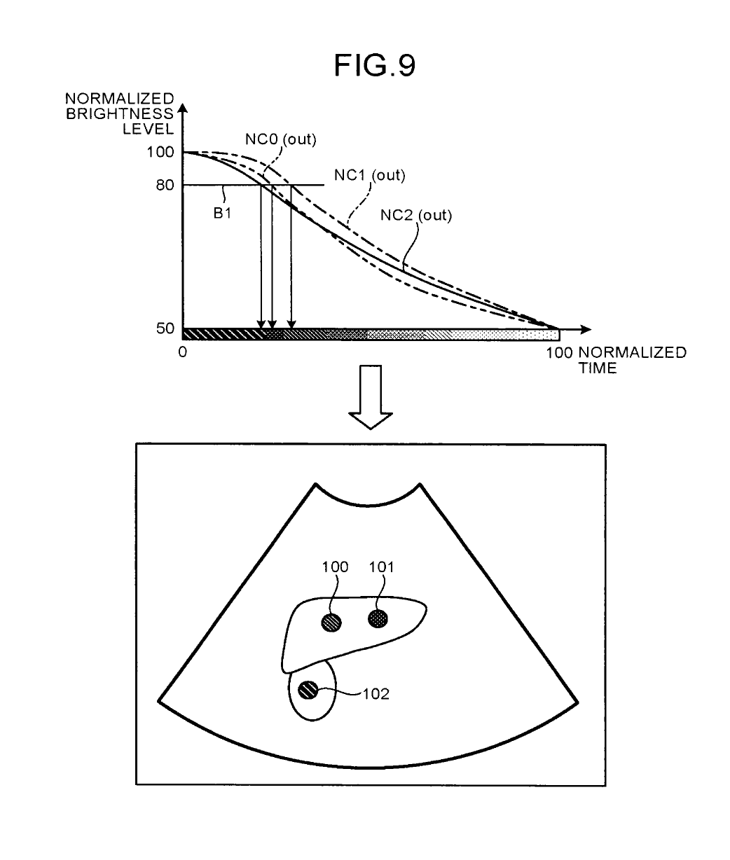

When the parametric imaging is set as one of the display modes that use an image, the transition image generating unit 153 illustrated in FIG. 1 performs the processes described below, according to an instruction from the controlling unit 18: The transition image generating unit 153 generates transition image data in which the tones are varied in accordance with the values of the parameters. After that, as one of the display modes that use an image, the controlling unit 18 causes the monitor 2 to display the transition image data. In the present embodiment, generating and displaying the transition image data is set as one of the display modes that use an image. Accordingly, the transition image generating unit 153 generates the transition image data by using the parameter obtained from each of the plurality of normalized curves. Next, the transition image data generated by the transition image generating unit 153 will be explained, with reference to FIGS. 9 to 11. FIGS. 9 to 11 are drawings for explaining the transition image generating unit.

When imaging the parameters related to the contrast agent inflow or the contrast agent outflow, the transition image generating unit 153 generates the transition image data by using a correspondence map (a time color map) in which mutually-different tones are associated with the normalized time on the normalized time axis. For example, the time color map is stored in the internal storage unit 17, in advance. FIG. 9 illustrates an example in which the normalized time is imaged as the parameter related to the contrast agent outflow, by using the normalized curves NC0(out), NC1(out), and NC2(out) illustrated in FIG. 7.

For example, as illustrated in the top section of FIG. 9, the controlling unit 18 causes the monitor 2 to display the normalized curves NC0(out), NC1(out), and NC2(out). Further, the controlling unit 18 causes the monitor 2 to further display a slide bar B1 with which the operator is able to set an arbitrary normalized brightness level. As illustrated in the top section of FIG. 9, the slide bar B1 is a line that is parallel to the normalized time axis and is orthogonal to the normalized brightness axis. Further, as illustrated in the top section of FIG. 9, the controlling unit 18 causes the time color map to be displayed on the normalized time axis, by using the same scale as the scale of the normalized time axis. The position and the scale for displaying the time color map may arbitrarily be changed.

After that, as illustrated in the top section of FIG. 9, for example, the operator moves the slide bar B1 to the position corresponding to the normalized brightness level “80”. The analyzing unit 152 obtains a normalized time corresponding to the normalized brightness level “80” from each of the curves NC0(out), NC1(out), and NC2(out). After that, the analyzing unit 152 determines the normalized time obtained from NC0(out) to be the parameter for the analysis region 100, determines the normalized time obtained from NC1(out) to be the parameter for the analysis region 101, and determines the normalized time obtained from NC2(out) to be the parameter for the analysis region 102 and subsequently notifies the transition image generating unit 153 of the determined parameters.

As illustrated in the bottom section of FIG. 9, the transition image generating unit 153 obtains a tone corresponding to the normalized time obtained from NC0(out) by referring to the time color map and colors the analysis region 100 in the ultrasound image data by using the obtained tone. Further, as illustrated in the bottom section of FIG. 9, the transition image generating unit 153 obtains a tone corresponding to the normalized time obtained from NC1(out) by referring to the time color map and colors the analysis region 101 in the ultrasound image data by using the obtained tone. In addition, as illustrated in the bottom section of FIG. 9, the transition image generating unit 153 obtains a tone corresponding to the normalized time obtained from NC2(out) by referring to the time color map and colors the analysis region 102 in the ultrasound image data by using the obtained tone. The ultrasound image data colored by using the tones obtained from the time color map is, for example, the ultrasound image data in which the analysis regions 100 to 102 are set.

Subsequently, the controlling unit 18 causes the monitor 2 to display the transition image data illustrated in the bottom section of FIG. 9. The transition image data is such data in which the outflow time is normalized and imaged for each of the analysis regions, so as to indicate the time it takes for the amount of the contrast agent that is present to decrease from the maximum amount to the predetermined percentage of the maximum amount, during the contrast agent outflow process.

In accordance with the moves of the slide bar B1 made by the operator, the analyzing unit 152 obtains an updated parameter of each of the analysis regions, whereas the transition image generating unit 153 updates and generates transition image data. The normalized brightness level may be set by using an arbitrary method, such as a method by which the operator inputs a numerical value. Alternatively, the present embodiment is also applicable to a situation where, for example, transition image data is generated and displayed as a moving image, as the value of the normalized brightness level is automatically changed.

When the parameters (the normalized times) related to the contrast agent inflow are to be imaged, processes are similarly performed by using the normalized curves NC0(in), NC1(in), and NC2(in) illustrated in FIG. 6, for example. The transition image data that is generated and displayed in that situation is such data in which the inflow time is normalized and imaged for each of the analysis regions, so as to indicate the time it takes for the amount of the contrast agent that is present to increase from the predetermined percentage of the maximum amount to the maximum amount, during the contrast agent inflow process.

Further, when imaging the parameters related to the contrast agent inflow or the contrast agent outflow, the transition image generating unit 153 generates the transition image data by using a correspondence map (a brightness color map) in which mutually-different tones are associated with the normalized brightness levels on the normalized brightness axis. For example, the brightness color map is stored in the internal storage unit 17, in advance. FIG. 10 illustrates an example in which the normalized brightness levels are imaged as the parameter related to the contrast agent outflow, by using the normalized curves NC0(out), NC1(out), and NC2(out) illustrated in FIG. 7.

For example, as illustrated in the top section of FIG. 10, the controlling unit 18 causes the monitor 2 to display the normalized curves NC0(out), NC1(out), and NC2(out). Further, the controlling unit 18 causes the monitor 2 to further display a slide bar B2 with which the operator is able to set an arbitrary normalized time. As illustrated in the top section of FIG. 10, the slide bar B2 is a line that is parallel to the normalized brightness axis and is orthogonal to the normalized time axis. Further, as illustrated in the top section of FIG. 10, the controlling unit 18 causes the brightness color map to be displayed on the normalized brightness axis, by using the same scale as the scale of the normalized brightness axis. The position and the scale for displaying the brightness color map may arbitrarily be changed.

After that, as illustrated in the top section of FIG. 10, for example, the operator moves the slide bar B2 to the position corresponding to the normalized time “60”. The analyzing unit 152 obtains a normalized brightness level corresponding to the normalized time “60” from each of the curves NC0(out), NC1(out), and NC2(out). After that, the analyzing unit 152 determines the normalized brightness level obtained from NC0(out) to be the parameter for the analysis region 100, determines the normalized brightness level obtained from NC1(out) to be the parameter for the analysis region 101, and determines the normalized brightness level obtained from NC2(out) to be the parameter for the analysis region 102 and subsequently notifies the transition image generating unit 153 of the determined parameters.

As illustrated in the bottom section of FIG. 10, the transition image generating unit 153 obtains a tone corresponding to the normalized brightness level obtained from NC0(out) by referring to the brightness color map and colors the analysis region 100 in the ultrasound image data by using the obtained tone. Further, as illustrated in the bottom section of FIG. 10, the transition image generating unit 153 obtains a tone corresponding to the normalized brightness level obtained from NC1(out) by referring to the brightness color map and colors the analysis region 101 in the ultrasound image data by using the obtained tone. In addition, as illustrated in the bottom section of FIG. 10, the transition image generating unit 153 obtains a tone corresponding to the normalized brightness level obtained from NC2(out) by referring to the brightness color map and colors the analysis region 102 in the ultrasound image data by using the obtained tone. The ultrasound image data colored by using the tones obtained from the brightness color map is, for example, the ultrasound image data in which the analysis regions 100 to 102 are set.

Subsequently, the controlling unit 18 causes the monitor 2 to display the transition image data illustrated in the bottom section of FIG. 10. The transition image data is such data in which the outflow amount of the contrast agent flowing out of each of the analysis region is normalized and imaged at mutually-the-same points in time on the time axis normalizing the contrast agent outflow process.

In accordance with the moves of the slide bar B2 made by the operator, the analyzing unit 152 updates and obtains a parameter of each of the analysis regions, whereas the transition image generating unit 153 updates and generates transition image data. The normalized time may be set by using an arbitrary method, such as a method by which the operator inputs a numerical value. Alternatively, the present embodiment is also applicable to a situation where, for example, transition image data is generated and displayed as a moving image, as the value of the normalized time is automatically changed.

When the parameters (the normalized brightness levels) related to the contrast agent inflow are to be imaged, processes are similarly performed by using the normalized curves NC0(in), NC1(in), and NC2(in) illustrated in FIG. 6, for example. The transition image data that is generated and displayed in that situation is such data in which the inflow amount of the contrast agent flowing into each of the analysis regions is normalized and imaged at mutually-the-same points in time on the time axis normalizing the contrast agent inflow process.

When imaging the parameters related to the contrast agent inflow and the contrast agent outflow, the transition image generating unit 153 generates transition image data by using a third correspondence map obtained by mixing a first correspondence map (a first time color map) and a second correspondence map (a second time color map). In this situation, the first time color map is a map in which mutually-different tones in a first hue are associated with the normalized time on the normalized time axis before the normalized maximum time at the normalized maximum point. The second time color map is a map in which mutually-different tones in a second hue are associated with the normalized time on the normalized time axis after the normalized maximum time. For example, the first time color map is a bluish color map, whereas the second color map is a reddish color map. For example, the first time color map and the second time color map are stored in the internal storage unit 17, in advance. FIG. 11 illustrates an example in which the normalized time is imaged as the parameters related to the contrast agent inflow and the contrast agent outflow, by using the normalized curves NC0, NC1, and NC2 illustrated in FIG. 8.

For example, as illustrated in FIG. 11, the controlling unit 18 causes the monitor 2 to display the normalized curves NC0, NC1, and NC2. Further, the controlling unit 18 causes the monitor 2 to further display a slide bar B3 with which the operator is able to set an arbitrary normalized brightness level. As illustrated in FIG. 11, the slide bar B3 is a line that is parallel to the normalized time axis and is orthogonal to the normalized brightness axis. Further, as illustrated in FIG. 11, the controlling unit 18 causes the first time color map and the second time color map to be displayed on the normalized time axis, by using the same scale as the scale of the normalized time axis. In FIG. 11, the normalized maximum time is at “0”, while the first time color map is displayed while being scaled at “−100 to 0” on the normalized time axis, whereas the second color map is displayed while being scaled at “0 to 100” on the normalized time axis. The position and the scale for displaying the first and the second time color maps may arbitrarily be changed.

After that, as illustrated in FIG. 11, for example, the operator moves the slide bar B3 to the position corresponding to the normalized brightness level “65”. The analyzing unit 152 obtains two normalized times (a negative normalized time and a positive normalized time) corresponding to the normalized brightness level “65” from each of the curves NC0, NC1, and NC2. After that, the analyzing unit 152 determines the two normalized times obtained from NC0 to be the parameters for the analysis region 100, determines the two normalized times obtained from NC1 to be the parameters for the analysis region 101, and determines the two normalized times obtained from NC2 to be the parameters for the analysis region 102 and subsequently notifies the transition image generating unit 153 of the determined parameters.

As illustrated in FIG. 11, the transition image generating unit 153 obtains a tone corresponding to the negative normalized brightness level obtained from NC0 by referring to the first time color map and obtains a tone corresponding to the positive normalized brightness level obtained from NC0 by referring to the second time color map. Further, as illustrated in FIG. 11, the transition image generating unit 153 colors the analysis region 100 in the ultrasound image data by using a tone resulting from mixing the two obtained tones together.

The transition image generating unit 153 performs a similar tone obtaining process for the two normalized brightness levels obtained from NC1 and, as illustrated in FIG. 11, colors the analysis region 101 in the ultrasound image data by using a tone resulting from mixing the two obtained tones together. Further, the transition image generating unit 153 performs a similar tone obtaining process for the two normalized brightness levels obtained from NC2 and, as illustrated in FIG. 11, colors the analysis region 102 in the ultrasound image data by using a tone resulting from mixing the two obtained tones together.

Subsequently, the controlling unit 18 causes the monitor 2 to display the transition image data generated by using FIG. 11. The transition image data is such data in which “the outflow time it takes for the amount of the contrast agent that is present to decrease from the maximum amount to the predetermined percentage of the maximum amount” and “the inflow time it takes for the amount of the contrast agent that is present to increase from the predetermined percentage of the maximum amount to the maximum amount” are normalized for each of the analysis regions, so that these normalized times are imaged at the same time.

In accordance with the moves of the slide bar B3 made by the operator, the analyzing unit 152 updates and obtains a parameter of each of the analysis regions, whereas the transition image generating unit 153 updates and generates transition image data. The normalized time may be set by using an arbitrary method, such as a method by which the operator inputs a numerical value. Alternatively, the present embodiment is also applicable to a situation where, for example, transition image data is generated and displayed as a moving image, as the value of the normalized time is automatically changed. Further, the present embodiment is also applicable to a situation where a two-dimensional time color map obtained by mixing the first time color map and the second time color map together is used. Furthermore, the present embodiment is also applicable to a situation where a time color map corresponding to the values of normalized time widths is simply used, instead of mixing the two time color maps together.

Further, when imaging the parameters related to the contrast agent inflow and the contrast agent outflow, the transition image generating unit 153 may generate transition image data by performing the following processes: The transition image generating unit 153 generates transition image data by using a first brightness color map and a second brightness color map. The first brightness color map is a first correspondence map in which mutually-different tones in a first hue are associated with the normalized brightness levels on the normalized brightness axis before the normalized maximum time at the normalized maximum point. The second brightness color map is a second correspondence map in which mutually-different tones in a second hue are associated with the normalized brightness levels on the normalized brightness axis after the normalized maximum time.

In that situation, the analyzing unit 152 obtains two normalized brightness levels corresponding to two specified normalized times “−T and +T” from each of the normalized curves. After that, the transition image generating unit 153 obtains a tone corresponding to the normalized brightness level at “−T” by referring to the first brightness color map, obtains a tone corresponding to the normalized brightness level at “+T” by referring to the second brightness color map, and further mixes the two obtained tones together. Thus, the transition image generating unit 153 generates the transition image data. The processes described above are also applicable to a situation where a two-dimensional brightness color map obtained by mixing the first brightness color map and the second brightness color map together is used. Furthermore, the present embodiment is also applicable to a situation where a brightness color map corresponding to the values of normalized brightness widths is simply used, instead of mixing the two brightness color maps together.

When the setting is made as illustrated in FIG. 3 or FIG. 4, one brightness transition curve is generated for mutually-the-same analysis region from each of the pieces of time-series data acquired during the two mutually-different times, so that two normalized curves are generated. In that situation, the transition image generating unit 153 arranges two identical pieces of ultrasound image data side by side and colors the analysis region in each of the pieces of ultrasound image data by using the tone corresponding to the normalized parameter obtained from the corresponding one of the normalized curves.

In another example, one brightness transition curve may be generated for mutually-the-same analysis region from each of the pieces of time-series data acquired during three mutually-different times, so that three normalized curves are generated. In that situation, the transition image generating unit 153 arranges three identical pieces of ultrasound image data side by side and colors the analysis region in each of the pieces of ultrasound image data by using the tone corresponding to the normalized parameter obtained from the corresponding one of the normalized curves.

In yet another example, a brightness transition curve may be generated for mutually-the-same two analysis regions from each of the pieces of time-series data acquired during two mutually-different times, so that two normalized curves are generated for each of the two times. In that situation, the transition image generating unit 153 arranges two identical pieces of ultrasound image data side by side and colors each of the two analysis regions in each of the pieces of ultrasound image data by using the tones corresponding to the two normalized times obtained from the corresponding one of the normalized curves.

In yet another example, in a situation where a brightness transition curve is generated for one or more mutually-the-same analysis regions from each of the pieces of time-series data acquired during two mutually-different times, the transition image generating unit 153 may generate a piece of transition image data by varying the tone in accordance with the ratio between the normalized parameters obtained from the normalized curves.

The present embodiment may also be configured in such a manner that, as the operator observes the transition image data and specifies an analysis region colored in accordance with the value of the normalized parameter, the value of the normalized parameter is displayed in the analysis region or near the analysis region. Further, the present embodiment may also be configured in such a manner that the analysis region is colored in accordance with the value of the normalized parameter, and also, that ultrasound image data rendering the value of the normalized parameter by using text in the analysis region or near the analysis region is generated and displayed as transition image data. Furthermore, the present embodiment may also be configured in such a manner that, without coloring the analysis region, ultrasound image data rendering the value of the normalized parameter by using text in the analysis region or near the analysis region is generated and displayed as transition image data.

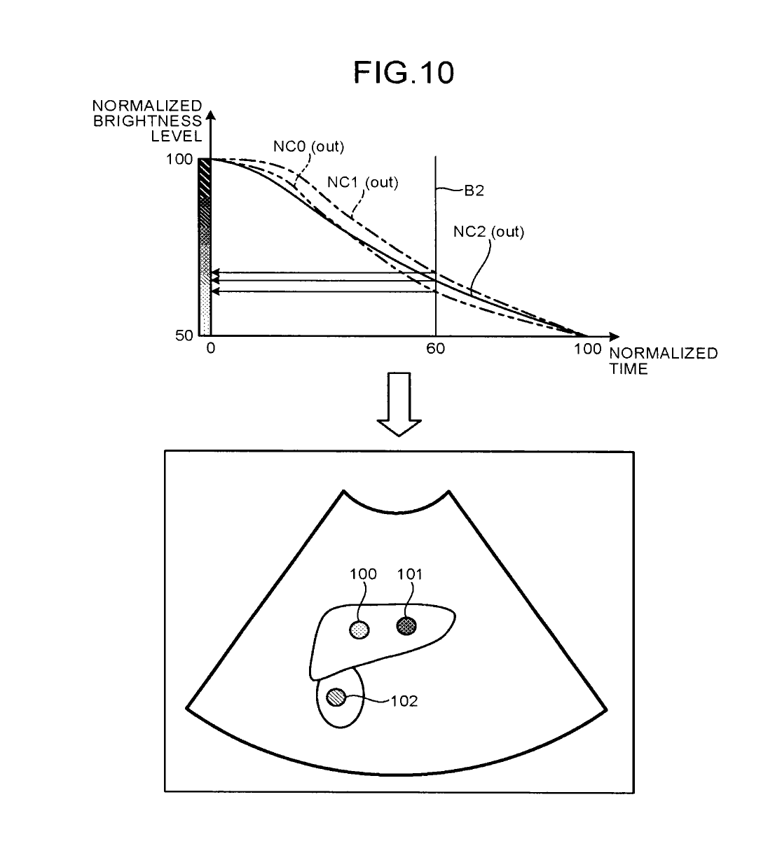

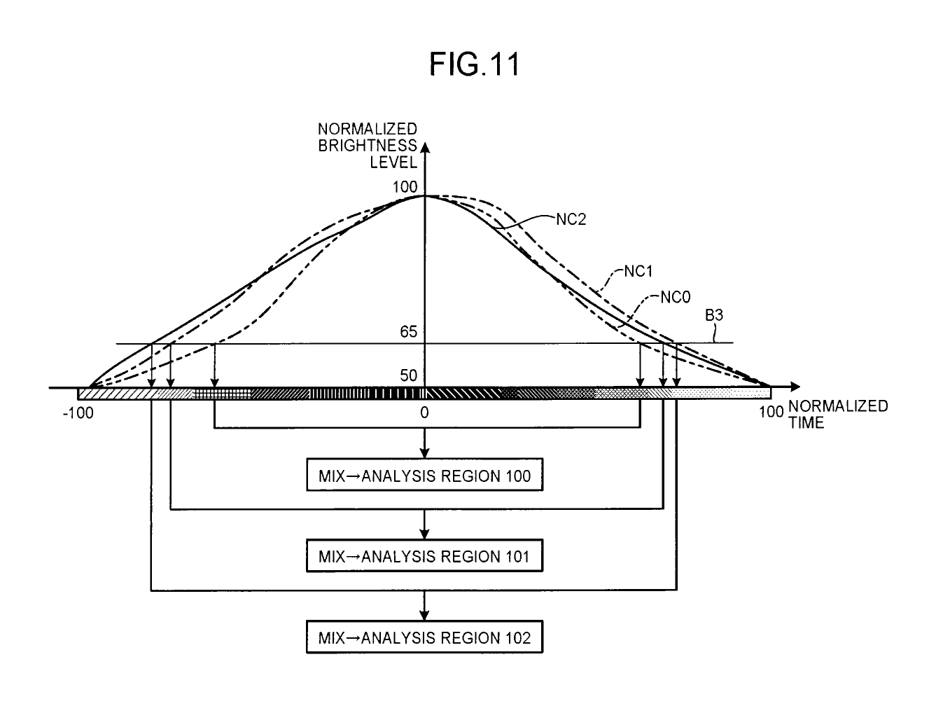

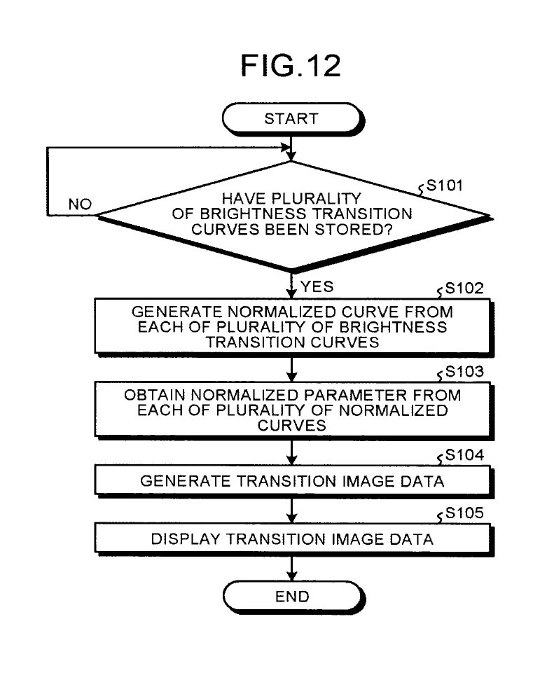

Next, exemplary processes performed by the ultrasound diagnostic apparatus according to the present embodiment will be explained, with reference to FIG. 12. FIG. 12 is a flowchart of the exemplary processes performed by the ultrasound diagnostic apparatus according to the present embodiment. FIG. 12 is a flowchart indicating the processes that are performed when the setting of an analysis region and the acquisition of a group of contrast enhanced image data have been completed, so that the generation of brightness transition curves has been started. The flowchart indicates the processes that are performed when transition image data is set as a display mode of the parameters.

As illustrated in FIG. 12, the analyzing unit 152 included in the ultrasound diagnostic apparatus according to the present embodiment judges whether the image memory 16 has stored a plurality of brightness transition curves (step S101). If the plurality of brightness transition curves have not been stored in the image memory 16 (step S101: No), the analyzing unit 152 stands by until the plurality of brightness transition curves are stored.

On the contrary, if the plurality of brightness transition curves have been stored in the image memory 16 (step S101: Yes), the analyzing unit 152 analyzes the shape characteristics and generates a normalized curve from each of the plurality of brightness transition curves (step S102). After that, the analyzing unit 152 obtains a normalized parameter from each of the plurality of normalized curves (step S103).

Subsequently, the transition image generating unit 153 obtains the tones corresponding to the values of the obtained parameters from the correspondence map and generates transition image data (step S104). After that, under the control of the controlling unit 18, the monitor 2 displays the transition image data (step S105), and the process is ended.

As explained above, according to the present embodiment, the normalized curves are generated by analyzing the shape characteristics of the brightness transition curves served as the analysis targets. In other words, according to the present embodiment, regardless of the conditions (e.g., the image taking conditions of the time-series data and the position of the analysis region) under which the brightness transition curves served as the analysis targets are generated, the normalized curves are generated from the brightness transition curves by using mutually-the-same objective criteria (the maximum brightness level, the first ratio, and the second ratio). Further, according to the present embodiment, the parameters normalizing the contrast agent inflow amount and outflow amount and the parameters normalizing the contrast agent inflow time and outflow time are obtained from the normalized curves.

Further, according to the present embodiment, the parametric imaging related to the dynamics of the bloodstream is performed by using the normalized parameters. In other words, according to the present embodiment, the parametric imaging is performed by using the relative values obtained from the normalized curves as the parameters, unlike conventional parametric imaging in which absolute values obtained from brightness transition curves are used as the parameters. Consequently, according to the present embodiment, it is possible to analyze the reflux dynamics of the contrast agent by using the objective criteria. Further, according to the present embodiment, it is possible to have not only the inflow process of the contrast agent, but also the outflow process of the contrast agent imaged by using the normalized parameters.

Furthermore, according to the present embodiment, it is possible to relatively compare the reflux dynamics of the contrast agent in mutually-different analysis regions by performing the parametric imaging in which the normalized curves are used. For example, according to the present embodiment, by observing the transition image data explained with reference to FIGS. 9 and 10 and so on, the doctor is able to perform a differential diagnosis process on the tumor site and to assess the degree of abnormality of the tumor blood vessels, by comparing the reflux dynamics of the contrast agent in the tissue used as a reference (e.g., the portal vein or the kidney) with the reflux dynamics of the contrast agent in the tumor site.

Further, according to the present embodiment, by performing the parametric imaging that uses the normalized curves, it is possible to relatively compare the reflux dynamics of the contrast agent before and after the treatment in mutually-the-same analysis region. For example, according to the present embodiment, by observing the transition image data generated by setting the analysis region illustrated in FIG. 3, the doctor is able to judge the effect of the treatment using the angiogenesis inhibitor.

Further, according to the present embodiment, by performing the parametric imaging that uses the normalized curves, it is possible to relatively compare the reflux dynamics of the plurality of types of contrast agents having mutually-different characteristics, in mutually-the-same analysis region. For example, according to the present embodiment, by observing the transition image data generated by setting the analysis region illustrated in FIG. 4 and comparing the reflux dynamics of the contrast agent A that is easily taken into Kupffer cells with the reflux dynamics of the contrast agent B that is not so easily taken into Kupffer cells, the doctor is able to perform a differential diagnosis process on the tumor site and to assess the degree of abnormality of the tumor blood vessels.

### Modified Examples

The ultrasound diagnosis process according to the exemplary embodiments described above may be carried out in various modified examples other than the processes described above. In the following sections, various modified examples of the embodiments described above will be explained. The processes in the various modified examples explained below may be combined, in an arbitrary form, with any of the processes in the embodiments described above.

For example, in the exemplary embodiments described above, the example is explained in which the parameters are obtained by normalizing the reflux dynamics of the contrast agent in the analysis region with respect to the brightness levels and the time. In other words, in the example described above, the brightness transition curve is normalized with respect to both the time axis and the brightness axis. However, the analyzing unit 152 may obtain a parameter by normalizing the reflux dynamics of the contrast agent in the analysis region with respect to the time. In other words, the present embodiment is also applicable to a situation where normalizing process is not performed with respect to the brightness axis, but a normalizing process is performed with respect to the time axis so as to set a normalized time axis and to generate a normalized curve. In that situation, the analyzing unit 152 generates a normalized curve from the brightness transition curve, by scaling the time axis to the normalized curve, while keeping the brightness levels as those in the actual data. Further, the analyzing unit 152 obtains the brightness levels (the absolute brightness levels) corresponding to specified normalized times, so that the transition image generating unit 153 generates transition image data in which the tones are varied in accordance with the obtained brightness levels.

In another example, if instructed by the operator, the analyzing unit 152 may obtain a parameter by normalizing the reflux dynamics of the contrast agent in the analysis region with respect to the brightness levels. In other words, the present embodiment is also applicable to a situation where normalizing process is not performed with respect to the time axis, but a normalizing process is performed with respect to the brightness axis so as to set a normalized brightness axis and to generate a normalized curve. In that situation, the analyzing unit 152 generates a normalized curve from the brightness transition curve, by scaling the brightness axis to the normalized curve, while keeping the time as that in the actual data. Further, the analyzing unit 152 obtains the times (the absolute times) corresponding to specified normalized brightness levels, so that the transition image generating unit 153 generates transition image data in which the tones are varied in accordance with the obtained times.

In yet another modified example of the embodiments described above, the analyzing unit 152 may obtain the normalized curve as a parameter, so that the controlling unit 18 causes the monitor 2 to display the normalized curve in one of the display modes using an image. Because the normalized curve is such a curve that is obtained by normalizing the reflux dynamics of the contrast agent, the operator is also able to analyze the reflux dynamics of the contrast agent by using the objective criteria, by observing the normalized curve itself. Thus, for example, when having generated a plurality of normalized curves, the analyzing unit 152 outputs the plurality of normalized curves to the controlling unit 18 as parameters. Subsequently, the controlling unit 18 causes the monitor 2 to display the plurality of normalized curves.

In this modified example, the graphs illustrated in FIGS. 6 to 8 are displayed on the monitor 2. Alternatively, the normalized curves displayed as the parameters may be such a curve that is obtained by performing the normalizing process with respect to only one of the axes, as explained in the modified example above. In yet another modified example of the embodiments described above, the analyzing unit 152 may obtain a normalized curve as a parameter, as well as another parameter from the normalized curve. For example, the normalized curve and the transition image data explained in the embodiment above may be displayed at the same time as parameters.

In yet another modified example of the embodiments described above, the analyzing unit 152 may output one or more values obtained from the normalized curve to the controlling unit 18 as a parameter, so that the controlling unit 18 causes the monitor 2 to display the one or more values in either a table or a graph. In this modified example, the analyzing unit 152 obtains, from the normalized curve, one or more parameters corresponding to the parameters that are conventionally obtained from a brightness transition curve (an approximate curve). The normalized curve used in this modified example may be a curve normalized with respect to the two axes or may be a curve normalized with respect to only one of the two axes.

Next, typical parameters that are conventionally obtained from a brightness transition curve of an analysis region will be explained. Examples of conventional parameters include the maximum value of the brightness level (the maximum brightness level), the time it takes for the brightness level to reach the maximum value (the maximum brightness time), and a Mean Transit Time (MTT). The MTT is a time from a point in time when the brightness level reaches “50% of the maximum brightness level” after the contrast agent has flowed in to a point in time when the brightness level reaches “50% of the maximum brightness level” when the contrast agent has flowed out after the maximum brightness level.

Another example of conventional parameters is a slope, i.e., the derivative of a brightness transition curve at the point in time when the brightness level reaches “50% of the maximum brightness level” during the contrast agent inflow process. Other examples of conventional parameters include an “‘Area Wash In’ that is an area value obtained by calculating the integral of” the brightness levels in a brightness transition curve “over an integration period from the contrast agent inflow time to the maximum brightness time”, an “‘Area Wash Out’ that is an area value obtained by calculating the integral of” the brightness levels in a brightness transition curve “over an integration period from the maximum brightness time to the contrast agent outflow time”, and an “‘Area Under Curve’ that is an area value obtained by calculating the integral of” the brightness levels in a brightness transition curve “over an integration period from the contrast agent inflow time to the contrast agent outflow time”. The “Area Wash In” value indicates the total amount of contrast agent that is present in the analysis region during the contrast agent inflow time. The “Area Wash Out” value indicates the total amount of contrast agent that is present in the analysis region during the contrast agent outflow time. The “Area Under Curve” value indicates the total amount of contrast agent that is present in the analysis region from the inflow time to the outflow time of the contrast agent.

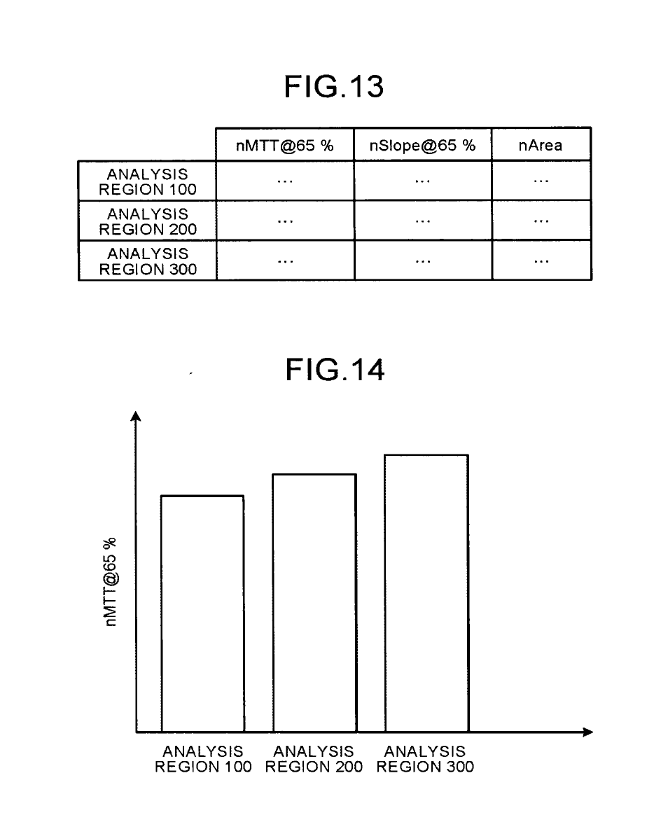

Next, an example will be explained in which the analyzing unit 152 obtains a “typical normalized parameter that makes it possible to objectively evaluate the reflux dynamics of the contrast agent” in each of the analysis regions 100, 200, and 300, by using the three normalized curves illustrated in FIG. 11. FIGS. 13 and 14 are drawings for explaining the modified example. For example, to obtain a normalized parameter corresponding to the conventional MTT, the analyzing unit 152 obtains a time (a normalized time) from the point in time when the normalized brightness level has increased to 65% of the normalized maximum brightness level “100” to the point in time when the normalized brightness level has decreased to 65% of the normalized maximum brightness level “100”. The analyzing unit 152 obtains this time as a normalized mean transit time for “65%” (nMTT@65%). The analyzing unit 152 obtains an “nMTT@65%” for each of the analysis regions 100, 200, and 300. The ratio used for calculating the normalized mean transit time may be changed to any arbitrary value other than 65%.

Further, for example, to obtain a normalized parameter corresponding to the conventional “slope” at the point in time when the brightness level reaches “50% of the maximum brightness level”, the analyzing unit 152 obtains the slope of the normalized curve at the time when the brightness level has become equal to 65% of the normalized maximum brightness level “100” during the contrast agent inflow process, as an “nSlope@65%”. The analyzing unit 152 obtains an “nSlope@65%” for each of the analysis regions 100, 200, and 300.

Further, for example, to obtain a normalized parameter corresponding to the conventional “Area Under Curve”, the analyzing unit 152 obtains an area value by calculating the integral of the normalized brightness levels in the normalized curve over the normalized time period “−100 to 100” as an “nArea”. The analyzing unit 152 obtains a “nArea” for each of the analysis regions 100, 200, and 300. Alternatively, the analyzing unit 152 may obtain an area value by calculating the integral of the normalized brightness levels in the normalized curve over the normalized time period “−100 to 0”, as a normalized parameter corresponding to the “Area Wash in”. Further, the analyzing unit 152 may obtain an area value by calculating the integral of the normalized brightness levels in the normalized curve over the normalized time period “0 to 100”, as a normalized parameter corresponding to the “Area Wash Out”.

Further, for example, as illustrated in FIG. 13, the controlling unit 18 converts the “nMTT@65%” of the analysis regions 100, 200, and 300, the “nSlope@65%” of the analysis regions 100, 200, and 300, and the “nArea” of the analysis regions 100, 200, and 300 into a table and causes the monitor 2 to display the table. The display mode in the format using a table is an example of a display mode in a format using text. Alternatively, for example, as illustrated in FIG. 14, the controlling unit 18 converts the “nMTT@65%” of the analysis regions 100, 200, and 300 into a bar graph and causes the monitor 2 to display the bar graph. Further, although not shown in the drawings, the controlling unit 18 also converts the other normalized parameters of the analysis regions 100, 200, and 300 into a bar graph and causes the monitor 2 to display the bar graphs. The display mode in the format using bar graphs is an example of a display mode in a format using text. By using these modified examples, it is also possible to analyze the reflux dynamics of the contrast agent by using the objective criteria.

In the modified examples above, the example is explained in which the analyzing unit 152 obtains the slope at the one point in time on the time axis of the normalized curve, as the normalized parameter. However, the analyzing unit 152 may obtain a slope at each of a plurality of points in time on the time axis of the normalized curve, as normalized parameters. In other words, the modified example described above may be configured so that the analyzing unit 152 calculates the derivative value at each of different normalized times on the normalized curve, as normalized parameters. In that situation, the controlling unit 18 causes the derivative values at the normalized times to be displayed as a table. Alternatively, the controlling unit 18 may generate a graph by plotting the derivative values at the normalized times and may cause the graph to be displayed.

In yet another modified example of the exemplary embodiments, a single brightness transition curve may be generated. In that situation, the analyzing unit 152 generates the normalized curve described above from the single brightness transition curve. After that, as explained in the exemplary embodiments and the modified examples, the controlling unit 18 causes the parameter to be displayed in various formats. For example, the controlling unit 18 causes the monitor 2 to display transition image data generated from a single normalized curve. In this modified example also, it is possible to analyze the reflux dynamics of the contrast agent by using the objective criteria. Further, because the image processing methods described above make it possible to analyze the reflux dynamics of the contrast agent by using the objective criteria, the image processing methods are applicable even to a situation where an analysis region is set in each of different subjects.

For example, the analyzing unit 152 generates a normalized curve A from the brightness transition curve of an analysis region that is set at a tumor site in the liver of a subject A. Further, for example, the analyzing unit 152 generates a normalized curve B from the brightness transition curve of an analysis region that is set at a tumor site in the liver of a subject B. It is preferable if the tumor sites of the two subjects are in substantially the same anatomical site. Further, for example, the transition image generating unit 153 generates transition image data A of the normalized curve A and generates transition image data B of the normalized curve B. Alternatively, for example, the analyzing unit 152 may calculate an nMTT(A) of the normalized curve A and an nMTT(B) of the normalized curve B. If the degrees of progression of the liver cancer are different between the subject A and the subject B, there is a high possibility that the values of the normalized parameters will be different. In other words, if the degrees of progression of the liver cancer are different between the subject A and the subject B, the patterns of the tones are different between the transition image data A and the transition image data B, and the values are different between nMTT(A) and nMTT(B). Thus, for example, the doctor is able to judge the difference in the degrees of progression in the liver cancer by comparing the transition image data A with the transition image data B.

Further, by using the method described above, it is possible to acquire a normalized parameter of each of a plurality of subjects whose degrees of progression of the liver cancer are different from one another and to put the acquired normalized parameters into a database. In that situation, when having obtained a new normalized parameter of a subject C having liver cancer, the doctor is able to determine the degree of progression of the subject C by referring to the database.

Further, in the description above, the example is explained in which the brightness transition curve being used is generated after the time-series data during the contrast enhanced time has been acquired. However, in yet another modified example of the exemplary embodiments, the brightness transition curve may be generated in a real-time manner while the time-series data during the contrast enhanced time is being acquired. In other words, the present embodiment is applicable to a situation where at least the imaging process or the like of the normalized parameter related to the contrast agent inflow is performed in a real-time manner, from the point in time when the maximum point in the brightness transition curve is obtained.

The image processing methods explained in any of the exemplary embodiments and the modified examples may be implemented by an image processing apparatus provided independently of the ultrasound diagnostic apparatus. The image processing apparatus is able to implement any of the image processing methods explained in the exemplary embodiments, by obtaining the time-series data acquired by performing the ultrasound scan on the subject P into whom the contrast agent has been administered. Alternatively, the image processing apparatus may implement any of the image processing methods described in the exemplary embodiments by obtaining the brightness transition curves.

Further, the constituent elements of the apparatuses that are illustrated in the drawings are based on functional concepts. Thus, it is not necessary to physically configure the elements as indicated in the drawings. In other words, the specific mode of distribution and integration of the apparatuses is not limited to the ones illustrated in the drawings. It is acceptable to functionally or physically distribute or integrate all or a part of the apparatuses in any arbitrary units, depending on various loads and the status of use. Further, all or an arbitrary part of the processing functions performed by the apparatuses may be realized by a Central Processing Unit (CPU) and a computer program that is analyzed and executed by the CPU or may be realized as hardware using wired logic.

Furthermore, the image processing methods explained in the exemplary embodiments and the modified examples may be realized by causing a computer such as a personal computer or a workstation to execute an image processing computer program (hereinafter, an “image processing program”) that is prepared in advance. The image processing program may be distributed via a network such as the Internet. Further, it is also possible to record the image processing program onto a computer-readable non-transitory recording medium such as a hard disk, a flexible disk (FD), a Compact Disk Read-Only Memory (CD-ROM), a Magneto-optical (MO) disk, a Digital Versatile Disk (DVD), or a flash memory (e.g., a Universal Serial Bus (USB) memory, a Secure Digital (SD) card memory), so that a computer is able to read the program from the non-transitory recording medium and to execute the read program.

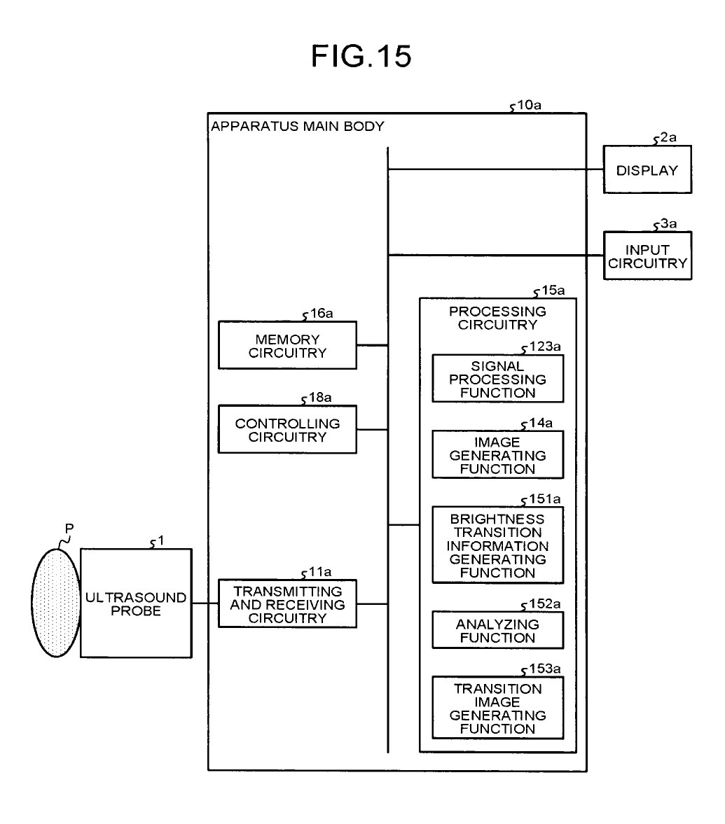

Another modified example of the ultrasound diagnostic apparatus that performs the above-described image processing methods will be explained with reference to FIG. 15. FIG. 15 is a block diagram of an exemplary configuration of the ultrasound diagnostic apparatus according to the modified example. The same components as those of the embodiments or the modified examples described above are indicated with the same symbols as those used in the embodiments or the modified examples. Detailed explanation will be omitted for the same content as that of the embodiments or the modified examples described above. The ultrasound diagnostic apparatus according to the present modified example includes the ultrasound probe 1, a display 2a, input circuitry 3a, and an apparatus main body 10a.

The display 2a corresponds to the monitor 2 illustrated in FIG. 1. The input circuitry 3a corresponds to the input device 3 illustrated in FIG. 1. The apparatus main body 10a corresponds to the apparatus main body 10 illustrated in FIG. 1.

The apparatus main body 10a includes transmitting and receiving circuitry 11a, processing circuitry 15a, memory circuitry 16a, and controlling circuitry 18a. The transmitting and receiving circuitry 11a corresponds to the transmitting and receiving unit 11 illustrated in FIG. 1. The memory circuitry 16a corresponds to the image memory 16 and the internal storage unit 17 illustrated in FIG. 1. That is, the memory circuitry 16a stores therein the same information as that stored in the image memory 16 and the internal storage unit 17. The controlling circuitry 18a corresponds to the controlling unit 18 illustrated in FIG. 1. That is, the controlling circuitry 18a performs the process performed by the controlling unit 18. The processing circuitry 15a is an example of the processing circuitry described in the claims. The controlling circuitry 18a is an example of the controlling circuitry described in the claims.

The processing circuitry 16a corresponds to the B-mode processing unit 12, the Doppler processing unit 13, the image generating unit 14, and the image processing unit 15 illustrated in FIG. 1. That is, the processing circuitry 16a performs the processes performed by the B-mode processing unit 12, the Doppler processing unit 13, the image generating unit 14, and the image processing unit 15. The process performed by the image processing unit 15 means the processes performed by the brightness transition information generating unit 151, the analyzing unit 152, and the transition image generating unit 153 illustrated in FIG. 1.

The processing circuitry 15a performs a signal processing function 123a, an image generating function 14a, a brightness transition information generating function 151a, an analyzing function 152a, and a transition image generating function 153a. The signal processing function 123a is a function implemented by the B-mode processing unit 12 and the Doppler processing unit 13 illustrated in FIG. 1. The image generating function 14a is a function implemented by the image generating unit 14 illustrated in FIG. 1. The brightness transition information generating function 151a is a function implemented by the brightness transition information generating unit 151 illustrated in FIG. 1. The analyzing function 152a is a function implemented by the analyzing unit 152 illustrated in FIG. 1. The transition image generating function 153a is a function implemented by the transition image generating unit 153 illustrated in FIG. 1.

The signal processing function 123a, the image generating function 14a, the brightness transition information generating function 151a, the analyzing function 152a, and the transition image generating function 153a that are performed by the processing circuitry 15a are stored in the memory circuitry 16a in the form of computer-executable programs, for example. The function of the controlling unit 18 performed by the controlling circuitry 18a is stored in the memory circuitry 16a in the form of a computer-executable program, for example. The processing circuitry 15a and the controlling circuitry 18a are processors that load programs from the memory circuitry 16a and execute the programs so as to implement the respective functions corresponding to the programs. That is, the processing circuitry 15a loading and executing the programs has the functions illustrated in FIG. 15. In the same manner, the controlling circuitry 18a loading and executing the program has the function performed by the controlling unit 18.

That is, the processing circuitry 15a loads a program corresponding to the signal processing function 123a from the memory circuitry 16a and executes the program so as to perform the same processes as those of the B-mode processing unit 12 and the Doppler processing unit 13. The processing circuitry 15a loads a program corresponding to the image generating function 14a from the memory circuitry 16a and executes the program so as to perform the same process as that of the image generating unit 14. The processing circuitry 15a loads a program corresponding to the brightness transition information generating function 151a from the memory circuitry 16a and executes the program so as to perform the same process as that of the brightness transition information generating unit 151. The processing circuitry 15a loads a program corresponding to the analyzing function 152a from the memory circuitry 16a and executes the program so as to perform the same process as that of the analyzing unit 152. The processing circuitry 15a loads a program corresponding to the transition image generating function 153a from the memory circuitry 16a and executes the program so as to perform the same process as that of the transition image generating unit 153. The controlling circuitry 18a loads a program corresponding to a function performed by the controlling unit 18 from the memory circuitry 16a and executes the program so as to perform the same process as that of the controlling unit 18.

Next, the correspondence between the modified example and the flowchart illustrated in FIG. 12 will be explained. Step S101 through Step S103 illustrated in FIG. 12 are implemented by the processing circuitry 15a loading the program corresponding to the analyzing function 152a from the memory circuitry 16a and executing the program. Step S104 illustrated in FIG. 12 is implemented by the processing circuitry 15a loading the program corresponding to the transition image generating function 153a from the memory circuitry 16a and executing the program. Step S105 illustrated in FIG. 12 is implemented by the processing circuitry 15a loading the program corresponding to the function performed by the controlling unit 18 from the memory circuitry 16a and executing the program.

Each of the above-described processors is, for example, a central processing unit (CPU), a graphics processing unit (GPU), an application specific integrated circuitry (ASIC), a programmable logic device (PLD), or a field programmable gate array (FPGA). The programmable logic device (PLD) is, for example, a simple programmable logic device (SPLD) or a complex programmable logic device (CPLD).

Each of the processors implements a function by loading and executing a corresponding program stored in the memory circuitry 16a. Instead of being stored in the memory circuitry 16a, a program may be install directly in the processors. In this case, each of the processors implements a function by loading and executing a corresponding program built directly in the processor.

The processors in the present modified example may not be separate from each other. For example, a plurality of processors may be combined as one processor that implements the respective functions. Alternatively, the components illustrated in FIG. 15 may be integrated into one processor that implements the respective functions.

The plurality of circuitry illustrated in FIG. 15 may be distributed or integrated as appropriate. For example, the processing circuitry 15a may be distributed as signal processing circuitry, image generating circuitry, brightness transition information generating circuitry, analyzing circuitry, and transition image generating circuitry that perform the signal processing function 123a, the image generating function 14a, the brightness transition information generating function 151a, the analyzing function 152a, and the transition image generating function 153a, respectively. Alternatively, for example, the controlling circuitry 18a may be integrated with the processing circuitry 15a.

As explained above, according to at least one aspect of the exemplary embodiments and the modified examples, it is possible to analyze the reflux dynamics of the contrast agent by using the objective criteria.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnostic apparatus comprising:
processing circuitry configured to generate brightness transition information indicating a temporal transition of a brightness level in an analysis region that is set in an ultrasound scan region, from time-series data acquired by performing an ultrasound scan on a subject to whom a contrast agent has been administered and to obtain a parameter by normalizing reflux dynamics of the contrast agent in the analysis region with respect to time, based on the brightness transition information; and
controlling circuitry configured to cause a display to display the parameter in a format using one or both of an image and text.

2. The ultrasound diagnostic apparatus according to claim 1, wherein the processing circuitry is configured to obtain the parameter by normalizing the reflux dynamics of the contrast agent in the analysis region with respect to either the brightness level or the brightness level and the time.

3. The ultrasound diagnostic apparatus according to claim 2, wherein the processing circuitry is configured to generate transition image data in which tones are varied in accordance with values of the parameter, and
the controlling circuitry is configured to cause the display unit to display the transition image data, as one of display modes using the image.

4. The ultrasound diagnostic apparatus according to claim 3, wherein the processing circuitry is configured to generate a brightness transition curve that is a curve indicating the temporal transition of the brightness level in the analysis region, as the brightness transition information and to generate a normalized curve from the brightness transition curve by normalizing either a time axis or a brightness axis and the time axis and performs one or both of a process to obtain the normalized curve as the parameter and a process to obtain the parameter from the normalized curve.

5. The ultrasound diagnostic apparatus according to claim 4, wherein the processing circuitry is configured to generate the normalized curve by using at least two points selected from: a maximum point at which the brightness level exhibits a maximum value in the brightness transition curve; a first point at which the brightness level reaches, before the maximum point, a first multiplied value obtained by multiplying the maximum value by a first ratio; and a second point at which the brightness level reaches, after the maximum point, a second multiplied value obtained by multiplying the maximum value by a second ratio.

6. The ultrasound diagnostic apparatus according to claim 5, wherein
the processing circuitry is configured to generate either a plurality of brightness transition curves respectively for a plurality of analysis regions that are set in the ultrasound scan region, or a plurality of brightness transition curves for at least one mutually-the-same analysis region set in mutually-the-same ultrasound scan region, respectively from a plurality of pieces of time-series data acquired by performing an ultrasound scan in mutually-the-same ultrasound scan region during a plurality of mutually-different periods and to generate the normalized curve from each of the plurality of brightness transition curves and performs one or both of a process to obtain each of the plurality of generated normalized curves as the parameter and a process to obtain the parameter from each of the plurality of normalized curves, and
if the transition image data is set as one of the display modes using the image, the transition image generating unit generates the transition image data by using the parameter obtained with respect to each of the plurality of normalized curves.

7. The ultrasound diagnostic apparatus according to claim 6, wherein
when obtaining the parameter related to a contrast agent inflow, the processing circuitry is configured to generate a plurality of normalized curves respectively from the plurality of brightness transition curves, by setting a normalized time axis and a normalized brightness axis on which the first points are plotted at a normalized first point that is mutually same among the brightness transition curves and on which the maximum points are plotted at a normalized maximum point that is mutually same among the brightness transition curves,
when obtaining the parameter related to a contrast agent outflow, the processing circuitry is configured to generate a plurality of normalized curves respectively from the plurality of brightness transition curves, by setting a normalized time axis and a normalized brightness axis on which the maximum points are plotted at a normalized maximum point that is mutually same on the brightness transition curves and on which the second points are plotted at a normalized second point that is mutually same among the brightness transition curves, and
when obtaining the parameter related to the contrast agent inflow and the contrast agent outflow, the processing circuitry is configured to generate a plurality of normalized curves respectively from the plurality of brightness transition curves, by setting a normalized time axis and a normalized brightness axis on which the first points, the maximum points, and the second points are plotted at the normalized first point, the normalized maximum point, and the normalized second point, respectively, among the brightness transition curves.

8. The ultrasound diagnostic apparatus according to claim 7, wherein, when imaging the parameter related to the contrast agent inflow or the contrast agent outflow, the processing circuitry is configured to generate the transition image data by using a correspondence map in which mutually-different tones are associated with normalized time on the normalized time axis.

9. The ultrasound diagnostic apparatus according to claim 7, wherein, when imaging the parameter related to the contrast agent inflow or the contrast agent outflow, the processing circuitry is configured to generate the transition image data by using a correspondence map in which mutually-different tones are associated with normalized brightness levels on the normalized brightness axis.

10. The ultrasound diagnostic apparatus according to claim 7, wherein, when imaging the parameter related to the contrast agent inflow and the contrast agent outflow, the processing circuitry is configured to generate the transition image data by using a first correspondence map in which mutually-different tones in a first hue are associated with normalized time on the normalized time axis before the normalized maximum time at the normalized maximum point and a second correspondence map in which mutually-different tones in a second hue are associated with normalized time after the normalized maximum time.

11. The ultrasound diagnostic apparatus according to claim 4, wherein
the processing circuitry is configured to output one or more values obtained from the normalized curve to the controlling circuitry as the parameter, and
the controlling circuitry is configured to cause the display unit to display the one or more values in either a table or a chart.

12. The ultrasound diagnostic apparatus according to claim 11, wherein the processing circuitry is configured to obtain a slope of the normalized curve on the time axis as the parameter.

13. An image processing apparatus comprising:
processing circuitry configured to generate brightness transition information indicating a temporal transition of a brightness level in an analysis region that is set in an ultrasound scan region, from time-series data acquired by performing an ultrasound scan on a subject to whom a contrast agent has been administered and to obtain a parameter by normalizing reflux dynamics of the contrast agent in the analysis region with respect to time, based on the brightness transition information; and
controlling circuitry configured to cause a display to display the parameter in a format using one or both of an image and text.

14. An image processing method comprising:
a process performed by processing circuitry to generate brightness transition information indicating a temporal transition of a brightness level in an analysis region that is set in an ultrasound scan region, from time-series data acquired by performing an ultrasound scan on a subject into whom a contrast agent has been administered and to obtain a parameter by normalizing reflux dynamics of the contrast agent in the analysis region with respect to time, based on the brightness transition information; and
a process performed by controlling circuitry to cause a display to display the parameter in a format using one or both of an image and text.
